(19) United States
(12) Patent Application Publication
(10) Pub. No.: US 2026/0191778 A1
(43) Pub. Date: Jul. 9, 2026

(54) AEROSOLIZED LIPID NANOPARTICLES AND USES THEREOF

(71) Applicant: Board of Regents, The University of Texas System, Austin, TX (US)

(72) Inventors: Mae LEWIS, Austin, TX (US); Debadyuti GHOSH, Austin, TX (US)

(73) Assignee: Board of Regents, The University of Texas System, Austin, TX (US)

(21) Appl. No.: 19/129,635

(22) PCT Filed: Nov. 14, 2023
(86) PCT No.: PCT/US2023/079604

§ 371 Date: May 13, 2025

Related U.S. Application Data
(60) Provisional application No. 63/425,129, filed on Nov. 14, 2022.

(51) Int. Cl. A61K 9/127; A61K 9/00; A61K 31/7105; A61K 47/18; A61K 47/24

(52) U.S. Cl. CPC A61K 9/127; A61K 9/0078; A61K 31/7105; A61K 47/18; A61K 47/24

(57) ABSTRACT

Lipid nanoparticles (LNPs) containing particular cationic ionizable lipids with a biologically active polynucleotides (e.g., RNAs) are provided. In some aspects, the LNP complexes are provided as aerosols and/or dry powders, such as for delivery to the lungs. Methods of making and using such compositions are provided.

### PRIORITY CLAIM

This application claims the benefit of priority to U.S. Provisional Application No. 63/425,129, filed on Nov. 14, 2022, the entire contents of which are hereby incorporated by reference.

### BACKGROUND OF THE INVENTION

### Field of the Invention

The present disclosure relates generally to the field of pharmaceutical formulation, biologics and the manufacture of the same. More particularly, lipid nanoparticles and method of using the same to deliver nucleic acid molecules.

### Description of Related Art

Messenger RNA (mRNA) is a promising therapeutic to treat pulmonary diseases. mRNA delivery has the potential to both treat and cure genetic diseases such as cystic fibrosis and primary ciliary dyskinesia (Translate Bio Inc., 2020; Woo et al., 2022). Preclinical results of mRNA delivery are promising, but a more efficacious carrier is needed to see clinical improvements (Translate Bio Inc., 2020).

Lipid nanoparticles (LNPs) have arisen as a powerful technology for delivering mRNA. For example, LNPs were the first drug carrier that became clinically approved in the U.S. to alleviate the COVID-19 pandemic (Baden et al., 2020; Polack et al., 2020). This versatile platform has shown success in delivering mRNA for many other applications like cancer, genetic diseases, and other infectious diseases (Zhang et al., 2020a; Qiu et al., 2021). However, targeting the lungs has been a challenge as these formulations naturally target the liver (Jayaraman et al., 2012; Akinc et al., 2019), and more particularly were made to deliver siRNA to the liver through intravenous route. More recently, they have shown efficacy in lung targeting systemic delivery. For example, a positively charged component had lung-targeting properties when delivered systemically (Cheng et al., 2020). In addition, modifications to a lipidoid head shifted the tropism from liver to lung (Qiu et al., 2022). While these therapies are promising, inhaled therapies for the lung offer increased concentration to the site of action with less off-target effects compared to systemic delivery (Rudokas et al., 2016). In addition, local delivery through aerosolization is attractive as it avoids off toxicity effects and delivers a high concentration of mRNA (Cipolla et al., 2013; Garbuzenko et al., 2009). To date, there has only been one completed clinical trial for the delivery of mRNA LNPs to the lungs (Translate Bio Inc., 2020). The Phase 1/2 clinical trial for CF patients (RESTORE-CF) evaluating an LNP encapsulating CFTR mRNA showed safety and tolerability after repeated dosing but did not significantly improve lung function. Therefore, there is a need for a translatable mRNA LNP for pulmonary delivery.

Delivery efficacy is highly dependent on the route of administration. Therefore, a formulation for local delivery will have to overcome several different barriers. To make an inhalable medicine, the formulation must be aerosolized. During this process, the LNP will experience shear force that causes mRNA degradation and subsequent loss of delivery. In addition, the LNP must deposit in the pulmonary tree at clinically relevant locations and successfully undergo endosomal escape for therapeutic efficacy. Then, LNPs must penetrate through the lung mucus and release their cargo into the cytosol to be translated via endosomal escape in the lung epithelial cells. While there have been studies addressing stability during aerosolization and mRNA delivery (Lokugamage et al., 2021; Kim et al., 2022), new compounds, compositions and methods are needed to overcome these barriers for local lung delivery.

### SUMMARY OF THE INVENTION

The present disclosure provides pharmaceutical compositions comprising a cationic ionizable lipids that can be used to deliver nucleic acids to therapeutically relevant locations.

In some embodiments, the present disclosure provides pharmaceutical compositions comprising:
a) a lipid nanoparticle (LNP); wherein the LNP comprises:
   i) a cationic ionizable lipid (CIL); wherein the cationic ionizable lipid is of the formula:

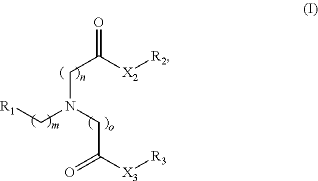

wherein:
   m, n, and o are each independently 1, 2, 3, 4, 5, 6, 7, 8, 9, or 10;
   X₂ and X₃ are each independently —O— or —N—;
   R₁ is hydroxy, amino, halo, or mercapto; or alkoxy_{(C≤8)}, alkylamino_{(C≤8)}, dialkylamino_{(C≤12)}, or a substituted version of any of these groups; and
   R₂ and R₃ are each independently alkyl_{(C≤24)}, alkenyl_{(C≤24)}, or alkynyl_{(C≤24)};
or a pharmaceutically available salt thereof;
ii) a phospholipid;
iii) a PEG-lipid; and
iv) a sterol; and
b) a nucleic acid;
wherein the composition is formulated for aerosolization. In further embodiments, the cationic ionizable lipid is further defined as:

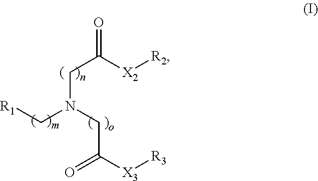

wherein:
   m is 1, 2, 3, 4, or 5;
   n and o are each independently 1, 2, 3, 4, 5, 6, 7, 8, 9, or 10;
   X₂ and X₃ are each independently —O— or —N—;
   R₁ is hydroxy, amino, halo, or mercapto; or alkoxy_{(C≤8)}, alkylamino_{(C≤8)}, dialkylamino_{(C≤12)}, or a substituted version of any of these groups; and
   R₂ and R₃ are each independently alkyl_{(C≤24)}, alkenyl_{(C≤24)}, or alkynyl_{(C≤24)};
or a pharmaceutically available salt thereof. In some embodiments, the cationic ionizable lipid is further defined as:

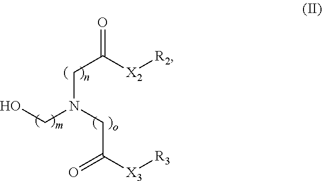

wherein:
   m is 1, 2, 3, 4, or 5;
   n and o are each independently 1, 2, 3, 4, 5, 6, 7, 8, 9, or 10; and
   X₂ and X₃ are each independently —O— or —N—; and
   R₂ and R₃ are each independently alkyl_{(C≤24)}, alkenyl_{(C≤24)}, or alkynyl_{(C≤24)}; or a pharmaceutically available salt thereof. In some embodiments, the cationic ionizable lipid is further defined as:

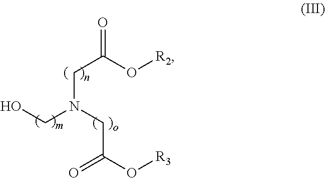

wherein:
   m is 1, 2, 3, 4, or 5;
   n and o are each independently 1, 2, 3, 4, 5, 6, 7, 8, 9, or 10; and
   R₂ and R₃ are each independently alkyl_{(C≤24)}, alkenyl_{(C≤24)}, or alkynyl_{(C≤24)};
or a pharmaceutically available salt thereof.

In some embodiments, the present disclosure provides pharmaceutical compositions wherein m is 1, 2, 3, or 4. In some embodiments, m is 2 or 3. In further embodiments, m is 2. In other embodiments, m is 3. In some embodiments, n is 3, 4, 5, 6, 7, 8, or 9. In further embodiments, n is 4, 5, 6, 7, or 8. In still further embodiments, n is 5 or 7. In yet further embodiments, n is 5. In other embodiments, n is 7. In some embodiments, o is 3, 4, 5, 6, 7, 8, or 9. In further embodiments, o is 4, 5, 6, 7, or 8. In still further embodiments, o is 5 or 7. In yet further embodiments, o is 5. In other embodiments, o is 7. In some embodiments wherein n is 5 and o is 7. In other embodiments, n and o are each 5. In still other embodiments, n is 7 and o is 5. In yet other embodiments, n and o are each 7.

In some embodiments, R₂ is alkyl_{(C≤24)} or substituted alkyl_{(C≤24)}. In further embodiments, R₂ is alkyl_{(C≤24)}. In still further embodiments, R₂ is alkyl_{(C6-20)}. In yet further embodiments, R₂ is n-alkyl_{(C6-20)}. In some embodiments, R₂ is n-alkyl_{(C6-12)}. In further embodiments, R₂ is n-hexane, n-heptane, n-octane, n-nonane, n-decane, n-undecane, or n-dodecane. In some embodiments, the carbon atom of R₂ that is bonded to X₂ is a secondary carbon. In some embodiments, R₂ is heptadecan-9-yl.

In some embodiments, R₃ is alkyl_{(C≤24)} or substituted alkyl_{(C≤24)}. In some embodiments. R₃ is alkyl_{(C≤24)}. In further embodiments, R₃ is alkyl_{(C6-20)}. In still further embodiments, R₃ is n-alkyl_{(C6-20)}. In some embodiments, R₃ is n-alkyl_{(C6-12)}. In further embodiments, R₃ is n-hexane, n-heptane, n-octane, n-nonane, n-decane, n-undecane, or n-dodecane. In some embodiments, the carbon atom of R₃ that is bonded to X₃ is a secondary carbon. In some embodiments, R₃ is heptadecan-9-yl. In some embodiments, R₂ is n-alkyl_{(C6-20)} and the carbon of R₃ that is bonded to X₃ is a secondary carbon. In other embodiments, R₂ and R₃ is n-alkyl_{(C6-20)}. In still other embodiments, the carbon of R₂ that is bonded to X₂ is a secondary carbon and the carbon of R₃ that is bonded to X₃ is a secondary carbon.

In some embodiments, the present disclosure provides compositions wherein the cationic ionizable lipid is further defined as:

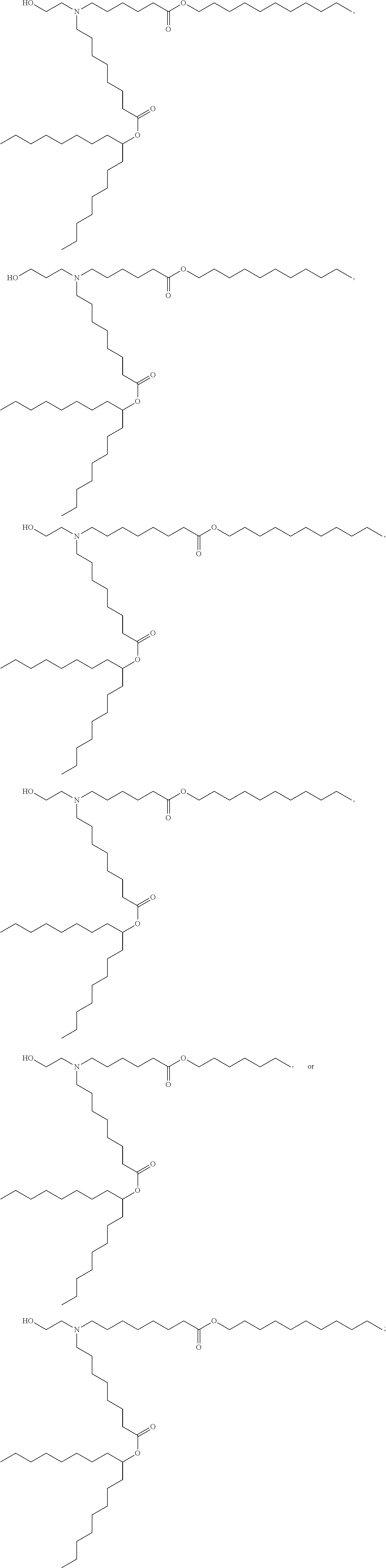

or a pharmaceutically acceptable salt thereof.

In some embodiments, the LNP of the presently disclosed compositions comprises a molar ratio of the cationic ionizable lipid to the LNP of from about 0.3-0.7. In further embodiments, the LNP comprises a molar ratio of the cationic ionizable lipid to the LNP of from about 0.4-0.6. In still further embodiments, the LNP comprises a molar ratio the cationic ionizable lipid to the LNP of about 0.45.

In some embodiments, the phospholipid comprises DOPE, DSPC, or DPPC. In further embodiments, the phospholipid is DPPC. In some embodiments, the LNP comprises a molar ratio of the phospholipid to the LNP of from about 0.02-0.4. In further embodiments, the LNP comprises a molar ratio of the phospholipid to the LNP of from about 0.05-0.3. In still further embodiments, the LNP comprises a molar ratio of the phospholipid to the LNP of about 0.2.

In some embodiments, the PEG-lipid comprises DMG-PEG, DMPE-PEG, or DSPE-PEG. In further embodiments, the PEG-lipid is DMPE-PEG. In some embodiments, the LNP comprises a molar ratio of the PEG-lipid to the LNP of from about 0.005-0.03. In further embodiments, the LNP comprises a molar ratio of the PEG-lipid to the LNP of from about 0.01-0.015. In still further embodiments, the LNP comprises a molar ratio of the PEG-lipid to the LNP of about 0.01. In some embodiments, the LNP comprises DOPE and DMG-PEG, DOPE and DMPE-PEG, DOPE and DSPE-PEG, DSPC and DMG-PEG, DSPC and DMPE-PEG, DSPC and DSPE-PEG, DPPC and DMG-PEG, DPPC and DMPE-PEG, or DPPC and DSPE-PEG. In further embodiments, the LNP comprises DPPC and DMPE-PEG.

In some embodiments, the sterol is cholesterol. In some embodiments, the LNP comprises a molar ratio of the sterol to the LNP of from about 0.1-0.6. In further embodiments, the LNP comprises a molar ratio of the sterol to the LNP of from about 0.15-0.5. In still further embodiments, the LNP comprises a molar ratio of the sterol to the LNP of about 0.34.

In some embodiments, the nucleic acid is a therapeutic nucleic acid. In some embodiments, the nucleic acid is siRNA, a miRNA, a pri-miRNA, a messenger RNA (mRNA), a cluster regularly interspaced short palindromic repeats (CRISPR) related nucleic acid, a single guide RNA (sgRNA), a CRISPR-RNA (crRNA), a trans-activating crRNA (tracrRNA), a plasmid DNA (pDNA), a transfer RNA (tRNA), an antisense oligonucleotide (ASO), a guide RNA, a double stranded DNA (dsDNA), a single stranded DNA (ssDNA), a single stranded RNA (ssRNA), and a double stranded RNA (dsRNA). In some embodiments, the nucleic acid is an RNA. In further embodiments, the nucleic acid comprises mRNA. In some embodiments, the nucleic acid comprises siRNA. In some embodiments, the nucleic acid is encapsulated in the LNP. In some embodiments, the ratio of the CIL to the nucleic acid is from about 20:1 to about 1:5 by weight. In further embodiments, the ratio of the CIL to the nucleic acid is from about 15:1 to about 1:3 by weight. In still further embodiments, the ratio of the CIL to the nucleic acid is about 11.3:1 by weight.

In some embodiments, the N/P ratio of the present compositions is from about 4-7. In further embodiments, the N/P ratio is from about 5-6. In still further embodiments, the N/P ratio is about 5.7. In some embodiments, the LNP has an average particle size of between about 50 nm and about 250 nm. In further embodiments, the LNP has an average particle size of between about 50 nm and about 150 nm. In still further embodiments, the composition has an average particle size of about 100 nm. In other embodiments, the composition has an average particle size of about 150 nm. In some embodiments, the composition has a polydispersity index (PDI) of between about 0.01 and about 0.5. In further embodiments, the composition has a polydispersity index (PDI) of between about 0.02 and about 0.4. In still further embodiments, the composition has a polydispersity index (PDI) of about 0.05. In other embodiments, the composition has a polydispersity index (PDI) of about 0.3. In some embodiments, the composition has a zeta potential of between about-0.5 mV and about-40 mV. In further embodiments, the composition has a zeta potential of between about-0.5 mV and about-20 mV. In still further embodiments, the composition has a zeta potential of about-10 mV. In other embodiments, the composition has a zeta potential of about-15 mV.

In one aspect, the present disclosure provides nebulized compositions in accordance with the pharmaceutical compositions described herein. In another aspect, the present disclosure provides methods of treating a disease, disorder, injury, or infection comprising administering an effective amount of pharmaceutical compositions described herein. In some embodiments, the disease is a genetic disease. In some embodiments, the disease, injury, or infection is a lung disease, a lung injury, or a lung infection. In further embodiments, the disease is a lung disease. In still further embodiments, the lung disease is interstitial lung disease, chronic obstructive pulmonary disease (COPD), asthma, cystic fibrosis (CF), pulmonary fibrosis, alpha-1 antitrypsin deficiency, or primary ciliary dyskinesia (PCD). In some embodiments, the pharmaceutical composition is formulated for administration via inhalation. In some embodiments, the subject is a mammal. In further embodiments, the subject is a human.

In another aspect, the present disclosure provides methods of modulating the expression of a gene comprising delivering a nucleic acid to a cell, the method comprising contacting the cell with pharmaceutical compositions disclosed herein under conditions sufficient to cause uptake of the nucleic acid into the cell. In some embodiments, the cell is contacted in vitro or ex vivo. In some embodiments, the cell is contacted in vitro. In some embodiments, the modulation of the gene expression is sufficient to treat a disease or disorder. In some embodiments, the nucleic acid is an mRNA.

Other objects, features, and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a block diagram detailing one embodiment of the lipid nanoparticle composition of the present disclosure

FIGS. 2A-2E show the in vitro delivery in ALI Calu-3 cells. (FIG. 2A) Compositions of set 1 LNPs. (FIG. 2B) Quantification of luminescence from ALI Calu-3 cells 24 hours after transfection with aerosolized set 1 LNPs delivering 1000 ng NLuc mRNA. Significance is relative to NLuc F11 (n=3; mean±standard deviation, **p<0.01, ***p<0.001; Student's t-test, double-tailed). (FIG. 2C) Structures of ionizable lipids used in sets 1 and 2 LNPs. (FIG. 2D) Compositions of set 2 LNPs. (FIG. 2E) Quantification of luminescence from ALI Calu-3 cells 24 hours after transfection with aerosolized set 2 and A-1 LNPs delivering 1000 ng NLuc mRNA (n=3; mean±standard deviation, **p<0.01, ***p<0.001; Student's t-test, double-tailed).

FIGS. 3A-3D show the physiochemical properties of non-aerosolized and aerosolized LNPs of the present disclosure. (FIG. 3A) Size (nm) by DLS (n=3; mean±standard deviation). (FIG. 3B) PDI by DLS (n=3; mean±standard deviation). (FIG. 3C) Zeta potential (mV) by DLS. (n=3; mean±standard deviation) (FIG. 3D) Encapsulation Efficiency (%) by Ribogreen. (n=2; mean±standard deviation)

FIG. 4 shows the TEM of NLuc F11, A-1, B-1, and NLuc Onpattro visualized at 43,000× before (top) and after (bottom) aerosolization. A-1 before aerosolization had a homogenous distribution of small and spherical particles. B-1 before aerosolization had a homogenous distribution of small and spherical particles. A-1 after aerosolization was still spherical but more polydisperse and larger in size. B-1 after aerosolization was more polydisperse, larger in size, and had more surface defects than A-1.

FIGS. 5A-5C present evidence that B-1 enables a significantly higher transfection efficiency compared to the baseline, NLuc F11, in Balb/c mice lungs. (FIG. 5A) Diagram of five mouse lung lobes in vivo and after harvesting. Created with BioRender.com. (FIG. 5B) Radiance in the five individual Balb/c mice lung lobes 24 hours after intratracheal administration of aerosolized LNPs delivering 750 ng NLuc mRNA. (FIG. 5C) Radiance quantification of FIG. 5A. (n=4; mean±standard deviation, **p<0.01, ***p<0.001; Student's t-test, double-tailed).

FIGS. 6A-6C show next generation impaction (NGI) results at a flow rate of 15 L/min. (FIG. 6A) NGI stages and droplet distribution in the human respiratory tract according to particle size. Created with BioRender.com. (FIG. 6B) Deposition profile across the device, throat, stages 1-7, and MOC (n=2; mean±standard deviation). (FIG. 6C) Aerodynamic performance (n=2; mean±standard deviation).

FIGS. 7A-7E show the impact of SM-102 analogs on LNP transfection. (FIG. 7A) Schematic of SM-102 structures relevant to the analogs. (FIG. 7B) SM-102 analog changes in structure compared to SM-102. (FIG. 7C) Quantification of luminescence from ALI Calu-3 cells 24 hours after transfection with aerosolized SM-102 analog LNPs delivering 1000 ng NLuc mRNA. Significance is relative to B-1 (n=3; mean±standard deviation, *p<0.05, **p<0.01, ***p<0.001, ns=not statistically significant; Student's t-test, double-tailed). (FIG. 7D) Deposition profile of C-1 compared to B-1 across the device, throat, stages 1-7, and MOC (n=2; mean±standard deviation). (FIG. 7E) Aerodynamic performance of C-1 compared to B-1 (n=2; mean±standard deviation).

FIGS. 8A-8B show in vitro delivery of non-aerosolized LNPs in ALI Calu-3 cells. Quantification of luminescence from ALI Calu-3 cells 24 hours after transfection with non-aerosolized (FIG. 8A) Set 1 and (FIG. 8B) Set 2 LNPs delivering 1000 ng NLuc mRNA (n=3; mean±standard deviation)

FIGS. 9A-9C show the assessment of cell types in the lungs for in vivo delivery. FIG. 9A: Schematic of Rosa26 locus in Ai9 mice and delivery schedule of Cre B-1 LNP. Upon successful Cre-mediated recombination, the stop cassette will be deleted, and the CAG promoter will drive expression of tdTomato. Aerosolized Cre B-1 LNP was delivered at 0.5 mg/kg once every other day over a period of four days. Lungs were harvested three days after the last dose. FIG. 9B: Quantification of tdTomato radiance from Ai9 mouse lungs after delivery of Cre B-1 compared to PBS control. FIG. 9C: Assessment of tdTomato cells in endothelial, epithelial, and immune cell populations using flow cytometry. (n=3; mean±standard deviation, **p<0.01; Student's t-test, double-tailed).

FIG. 10 shows the quantification of radiance in each individual lung lobe from FIG. 5B.

### DESCRIPTION OF ILLUSTRATIVE EMBODIMENTS

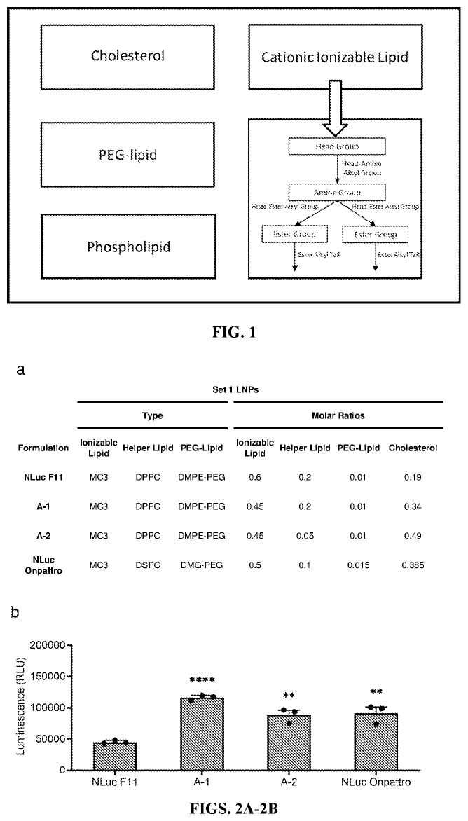

Provided herein are lipid nanoparticles (LNP) carriers for biologically active nucleic acid molecules. In some cases, LNPs of the embodiments can comprise single stranded or double stranded RNA or DNA. Such polynucleotides can be encapsulated in or in complex with lipid nanoparticle. For example, in some cases, polynucleotides, such as mRNAs are provided in complex with LNPs. In certain aspects, the LNPs comprise at least one cationic ionizable lipid, at least one phospholipid, at least one PEGylated lipid and cholesterol, such as depicted in FIG. 1. In certain aspects, the polynucleotide-LNP can be for gene replacement and/or gene editing. In some aspects, a mRNA-LNP complex can encode a therapeutically active protein (e.g., for gene replacement therapy) or an antigen (e.g., for vaccination). In preferred aspects, the LNP carriers and biologically active polynucleotides can be formulated as an aerosol (e.g., via nebulization) such as for delivery to lung. In further aspects, LNP complexes are provided in dry powders, such as by ultra-rapid freezing (URF). Preferably, such dry powder compositions additionally include at least a first excipient. For example, RNA-LNP powders are provided that further comprise at least a first excipient, such as sugar or amino acid. In some aspects, dry powders can be directly administered (e.g., by dispersion in the lungs) to subjects to treat a disease or stimulate an immune response. In some embodiments, the presently disclosed pharmaceutical compositions may exhibit improved delivery of a nucleic acid. The pharmaceutical composition may demonstrate less degradation by native RNAses, exhibit less targeting or tagging by antibodies and thus lower clearance by alveolar macrophages, or be less vulnerable to becoming trapped in the mucus and removed via mucociliary clearance. The present pharmaceutical compositions may, together with or in addition to any of the properties mentioned above, have a favorable transfection efficiency, particularly in lung cells.

### I. Nanoparticle and Nanoparticle Compositions

As used herein, the term “nanoparticle” refers to any material having dimensions in the 1-1,000 nm range. In some embodiments, nanoparticles have dimensions in the 50-500 nm range. Nanoparticles used in the present embodiments include such nanoscale materials as a lipid-based nanoparticle, a superparamagnetic nanoparticle, a nanoshell, a semiconductor nanocrystal, a quantum dot, a polymer-based nanoparticle, a silicon-based nanoparticle, a silica-based nanoparticle, a metal-based nanoparticle, a fullerene and a nanotube (Ferrari, 2005). The conjugation of polypeptide or nucleic acids to nanoparticles provides structures with potential application for targeted delivery, controlled release, enhanced cellular uptake and intracellular trafficking, and molecular imaging of therapeutic peptides in vitro and in vivo (West, 2004; Stayton et al., 2000; Ballou et al., 2004; Frangioni, 2003; Dubertret et al., 2002; Michalet et al., 2005; Dwarakanath et al., 2004.)

### (1) Lipid Nanoparticles (LNPs)

Lipid-based nanoparticles include liposomes, lipid preparations and lipid-based vesicles. Lipid-based nanoparticles may be positively charged, negatively charged or neutral. In preferred embodiments of the present disclosure, the lipid-based nanoparticles of the present disclosure comprise a cationic ionizable lipid.

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes and multivesicular liposomes. Liposomes provided herein may be positively charged, negatively charged or neutrally charged. In certain embodiments, the liposomes are neutral in charge.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In specific aspects, a polypeptide or nucleic acids may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

The size of a liposome varies depending on the method of synthesis. Liposomes in the present embodiments can be a variety of sizes. In certain embodiments, the liposomes are small, e.g., less than about 200 nm, about 190 nm, about 180 nm, about 170 nm, about 160 nm, about 150 nm, about 140 nm, about 130 nm, about 120 nm, about 110 nm, about 100 nm, about 90 nm, about 80 nm, about 70 nm, about 60 nm, or about 50 nm in external diameter. For example, in general, prior to the incorporation of nucleic acid, a liposome for use according to the present embodiments comprises a size of about 50 to 250 nm. Such liposome formulations may also be defined by particle charge (zeta potential) and/or optical density (OD). For instance, a liposome formulation will typically comprise an OD₄₀₀ of less than 0.45 prior to nucleic acid incorporation. Likewise, the overall charge of such particles in solution can be defined by a zeta potential of about 50-80 mV. In other embodiments, the liposomes or lipid nanoparticles may have a larger diameter, such as about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, or any range derivable therein.

The liposomes provided by the present disclosure are shown, for example, above in the summary of the invention section and in the claims below. They may be made using the methods outlined in the Examples section. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Furthermore, in preparing such liposomes, any protocol described herein, or as would be known to one of ordinary skill in the art may be used. Additional non-limiting examples of preparing liposomes are described in WO02/100435A1, WO03/015757A1, WO04029213A2, U.S. Application 2004/0208921, U.S. Pat. Nos. 4,728,578, 4,728,575, 4,737,323, 4,533,254, 4,162,282, 4,310,505, and 4,921,706; 5,030,453, 5,962,016, 6,680,068, International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference). A process of making liposomes is also described in WO04/002453A1.

In certain embodiments, the lipid-based nanoparticle is a positive liposome. “Positive liposomes” or “cationic liposomes”, as used herein, are defined as liposomes having one or more lipid components that yield an essentially positive net charge (substantially positive). By “essentially positive”, it is meant that overall, lipid components within a given population (e.g., a population of liposomes) include positive charges that are not canceled by an opposite charge of another component (i.e., fewer than 10% of components are canceled by an opposite charge of another component, more preferably fewer than 5%, and most preferably fewer than 1%). In certain embodiments, positive liposomes may include mostly lipids and/or phospholipids that are themselves positive under physiological conditions (i.e., at about pH 7). As used herein, the lipid components that yield an essentially positive net charge of the positive liposomes, that is, the lipids that are themselves positive under physiological conditions, may also be known as cationic ionizable lipids. In some embodiments, the cationic ionizable lipids may be neutral at physiological pH and positively charged in acidic pH or environments. The localized microenvironment surrounding cationic ionizable lipids may affect the protonation state of the cationic ionizable lipids and result in positively charged cationic ionizable lipids under conditions which would not otherwise be thought to result in positively charged cationic ionizable lipids. In some embodiments, the cationic ionizable lipid is an amino lipid. In some embodiments, the cationic ionizable lipids comprise a tertiary amine. In some embodiments, the alkyl groups attached to the tertiary amine may be independently substituted with functional groups, such as esters or hydroxy groups. In some embodiments, the cationic ionizable lipid is SM-102, MC3, or ALC-0315.

The cationic ionizable lipid component of lipid nanoparticle compositions of the present disclosure may be present in a variety of molar ratios with respect to the composition. In some embodiments of the present invention, the cationic ionizable lipid is present in a molar ratio with respect to the lipid nanoparticle composition of from about 0.2 to about 1.0. In some embodiments, the molar ratio of cationic ionizable lipid to lipid nanoparticle composition is from about 0.3 to about 0.7 or from about 0.4 to about 0.6. The molar ratio of cationic lipid to lipid nanoparticle composition may be about 0.2, about 0.25 about 0.3, about 0.35, about 0.4, about 0.45, about 0.5, about 0.55, about 0.6, about 0.65, about 0.7, about 0.75, about 0.8, about 0.85, about 0.9, about 0.95, or about 1.0, or any range derivable therein. In some embodiments, the molar ratio of cationic lipid to lipid nanoparticle composition is about 0.45.

The cationic ionizable lipids and other lipids of the present disclosure may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Cationic ionizable lipids may occur as racemates and racemic mixtures, single enantiomers, diastereomeric mixtures and individual diastereomers. In some embodiments, a single diastereomer is obtained. The chiral centers of the cationic ionizable lipids of the present disclosure can have the S or the R configuration. Furthermore, it is contemplated that one or more of the cationic ionizable lipids may be present as constitutional isomers. In some embodiments, the compounds have the same formula but different connectivity.

Chemical formulas used to represent cationic ionizable lipids of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given formula, and regardless of which one is most prevalent, all tautomers of a given chemical formula are intended.

The cationic ionizable lipids of the present disclosure may also have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the cationic ionizable lipids of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

It should be recognized that the particular anion or cation forming a part of any salt form of a cationic ionizable lipids provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### (2) Lipids

In some aspects of the present disclosure, one or more additional types of lipids are mixed with the cationic ionizable lipids of the instant disclosure to create a nanoparticle composition. In some embodiments, the cationic ionizable lipids are mixed with 1, 2, 3, 4, or 5 different types of lipids. It is contemplated that the cationic ionizable lipids can be mixed with multiple different lipids of a single type.

In some embodiments, at least one of the additional lipids may be a steroid or a steroid derivative. In some embodiments, the additional lipid may be a PEG lipid. In some embodiments, the additional lipid may be a phospholipid. In some embodiments, the nanoparticle composition comprises a steroid or a steroid derivative, a PEG lipid, and a phospholipid, or any combination thereof. Additional details of the types of lipids that may be used to form the nanoparticle composition are provided in the sections that follow.

### Steroids and Steroid Derivatives

In the lipid nanoparticle compounds of the present disclosure of the present disclosure, the cationic ionizable lipids (or compounds) are mixed with one or steroids or steroid derivatives and other components described below to form the nanoparticle composition. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula below:

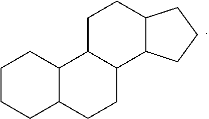 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

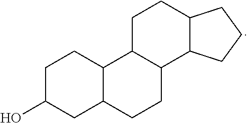 [SMILES: OC1CCC2C(CCC3C4CCCC4CCC23)C1]

Another steroid or steroid derivative is a cholestane or cholestane derivative. In a cholestane, the ring structure is further defined by the formula:

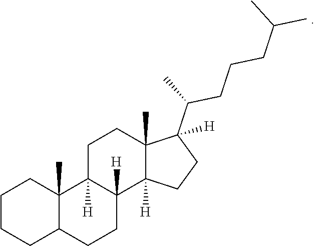

As described above, a cholestane derivative includes one or more non-alkyl substitution of the above ring system. The cholestane or cholestane derivative may be a cholestene or cholestene derivative or a sterol or a sterol derivative. The cholestane or cholestane derivative may be both both a cholestere and a sterol or a derivative thereof. In preferred embodiments, the nanoparticle composition comprises cholesterol.

In some embodiments, the present composition comprises a molar ratio of the steroid or steroid derivative to the lipid nanoparticle composition of from about 0.05 to about 0.12 or from about 0.1 to about 0.6. The molar ratio may be from about 0.15 to about 0.5 such as a molar ratio of about such as a molar ratio of steroid or steroid derivative to lipid nanoparticle composition of about 0.34. In some embodiments, the molar ratio of steroid or steroid derivative to lipid nanoparticle composition is about 0.05, about 0.10, about 0.15, about 0.20, about 0.25, about 0.30, about 0.35, about 0.4, about 0.45, about 0.50, about 0.55, about 0.60, about 0.65, about 0.70, about 0.75, about 0.80, about 0.85, about 0.90, about 1.0, or about 1.2, or any range derivable therein.

### PEG or PEGylated Lipid

In the lipid nanoparticle compounds of the present disclosure of the present disclosure, the cationic ionizable lipids (or compounds) are mixed with one or more PEGylated lipids (or PEG lipid) and other components described above and below to form the nanoparticle composition. In some embodiments, the present disclosure comprises using any lipid to which a PEG group has been attached. In some embodiments, the PEG lipid is a diglyceride which also comprises a PEG chain attached to the glycerol group. In other embodiments, the PEG lipid is a compound which contains one or more C6-C24 long chain alkyl or alkenyl group or a C6-C24 fatty acid group attached to a linker group with a PEG chain. In some embodiments, the PEG-lipid has an advantage such as that it prevents aggregation or reduces uptake of the composition by immune cells. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, the PEG lipid is PEG modified diastearoylphosphatidylethanolamine. In some embodiments, the PEG lipid comprises a PEG modified phospholipid, such as any of the lipids mentioned in the section that follows. In some embodiments, the PEG lipid is a PEG modified dimyristoyl phosphatidylethanolamine or a PEG modified myristoyl diglyceride.

In some embodiments, the PEG modification is measured by the molecular weight of PEG component of the lipid. In some embodiments, the PEG modification has a molecular weight from about 100 to about 5,000. In some embodiments, the molecular weight is from about 200 to about 500 or from about 1,200 to about 3,000. Some non-limiting examples of lipids that may be used in the present disclosure are taught by U.S. Pat. No. 5,820,873, WO 2010/141069, or U.S. Pat. No. 8,450,298, which is incorporated herein by reference.

In some embodiments, the present composition comprises a molar ratio of the PEG lipid to the lipid nanoparticle composition of from about 0.001 to about 0.04 or from about 0.005 to about 0.03. The molar ratio may be from about 0.01 to about 0.015. In some embodiments, the molar ratio of PEG lipid to the lipid nanoparticle composition may be about 0.01. In some embodiments, the ratio is about 0.001, about 0.005, about 0.006, about 0.007, about 0.008, about 0.009, about 0.01, about 0.011, about 0.012, about 0.013, about 0.014, about 0.015, about 0.02, about 0.025, about 0.03, about 0.035, to about 0.04 or any range derivable therein.

### Phospholipids

In the lipid nanoparticle compounds of the present disclosure of the present disclosure, the cationic ionizable lipids (or compounds) are mixed with one or more phospholipids and other components described above and below to form the nanoparticle composition. The phospholipids may also be referred to herein as “helper lipids.” In some embodiments, compositions disclosed herein comprise a helper lipid which comprises a phosphate group. In some embodiments, more than one kind of phospholipid may be used to form the composition. In some embodiments, the phospholipid is a structure which contains one or two long chain C6-C24 alkyl or alkenyl groups, a glycerol or a sphingosine, one or two phosphate groups, and, optionally, a small organic molecule. In some embodiments, the small organic molecule is an amino acid, a sugar, or an amino substituted alkoxy group, such as choline or ethanolamine. In some embodiments, the phospholipid is a phosphatidylcholine. In some embodiments, the phospholipid is distearoylphosphatidylcholine, dioleoylphosphatidylycholine or dipalmitoylphosphatidylcholine. In some embodiments, the helper lipid may be neutral under physiological conditions (i.e., at about pH 7). In some embodiments, the helper lipid has an advantage such as that it improves the structure or enhances endosomal escape.

Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating positive liposomes. In certain embodiments, the phospholipid DPPC is used to produce positive liposomes.

Phospholipids that may be components of compositions disclosed herein include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

In some embodiments, the present composition comprises a molar ratio of the phospholipid to the lipid nanoparticle composition from about 0.01 to about 0.5 or about 0.02 to about 0.4. The molar ratio may be from about 0.05 to about 0.3 such as a molar ratio of about 0.2. In some embodiments, the molar ratio of phospholipid to lipid nanoparticle composition is from about 0.01, about 0.03, about 0.05, about 0.07, about 0.09, about 0.1, about 0.12, about 0.14, about 0.16, about 0.18, about 0.2, about 0.22, about 0.24, about 0.26, about 0.28, about 0.3, about 0.35, to about 0.4, or any range derivable therein.

Phospholipids may be from natural or synthetic sources. However, phospholipids from natural sources, such as egg or soybean phosphatidylcholine, brain phosphatidic acid, brain or plant phosphatidylinositol, heart cardiolipin and plant or bacterial phosphatidylethanolamine are not used, in certain embodiments, as the primary phosphatide (i.e., constituting 50% or more of the total phosphatide composition) because this may result in instability and leakiness of the resulting liposomes.

### II. Biologically Active Polynucleotides

Methods and composition of the embodiments concern biologically active polynucleotides. In some cases, these can comprise single stranded or double stranded RNA or DNA. It should be clear that the present disclosure is not limited to the specific nucleic acids disclosed herein. The present disclosure is not limited in scope to any particular source, sequence, or type of nucleic acid, however, as one of ordinary skill in the art could readily identify related homologs in various other sources of the nucleic acid including nucleic acids from non-human species (e.g., mouse, rat, rabbit, dog, monkey, gibbon, chimp, ape, baboon, cow, pig, horse, sheep, cat and other species). It is contemplated that the nucleic acid used in the present disclosure can comprises a sequence based upon a naturally-occurring sequence.

The amount of nucleic acid encapsulated by or located within the lipid nanoparticle may vary based on the intended use. The amount of nucleic acid may be calculated as a ratio with respect to the lipid nanoparticle composition (w/w) or to any of the individual components of the lipid nanoparticle composition (w/w). For example, the ratio of cationic ionizable lipid to nucleic acid may be about from about 50:1 (w/w), about 20:1 (w/w), about 15:1 (w/w), about 14:1 (w/w), about 13:1 (w/w), about 12:1 (w/w), about 11:1 (w/w), about 10:1 (w/w), about 9:1 (w/w), about 8:1 (w/w), about 7:1 (w/w), about 6:1 (w/w), to about 5:1 (w/w), or any range derivable therein. In some embodiments, the ratio of cationic ionizable lipid to nucleic acid is about 11.33 (w/w). The length of the nucleic acid encapsulated by or located within the lipid nanoparticle may also vary based on the intended use. The length of the nucleic acid may be about 20 bp, about 50 bp, about 75 bp, about 100 bp, about 150 bp, about 200 bp, about 250 bp, about 300 bp, about 350 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1000 bp, or any range derivable therein. Longer nucleic acids are also contemplated, such as nucleic acids that are about 1500 bp, about 2000 bp, about 2500 bp, about 3000 bp, about 3500 bp, about 4000 bp, about 4500 bp, about 5000 bp, about 5500 bp, about 6000 bp, about 6500 bp, about 7000 bp, about 7500 bp, about 8000 bp, about 8500 bp, about 9000 bp, about 9500 bp, about 10,000 bp, or any range derivable therein.

In some aspects, the nucleic acid is a sequence which silences, is complimentary to, or replaces another sequence present in vivo. Sequences of 17 bases in length should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or longer are contemplated as well.

The nucleic acid used herein may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present disclosure may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

In some embodiments, the nucleic acid comprises one or more antisense segments which inhibits expression of a gene or gene product. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G: C) and adenine paired with either thymine (A: T) in the case of DNA, or adenine paired with uracil (A: U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to form a siRNA or to generate specific constructs. For example, where an intron is desired in the ultimate construct, a genomic clone will need to be used. The cDNA, siRNA, or a synthesized polynucleotide may provide more convenient restriction sites for the remaining portion of the construct and, therefore, would be used for the rest of the sequence. Other embodiments include dsRNA or ssRNA, which may be used to target genomic sequences or coding/non-coding transcripts.

In other embodiments, the nanoparticles may comprise a nucleic acid which comprises one or more expression vectors are used in a gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

mRNA

In some aspects, the present compounds and compositions may be used in the delivery of an mRNA to a cell. Messenger RNA or mRNA are short RNA strands which transfer the genetic code from the DNA to the ribosomes so the mRNA may be translated into a therapeutic protein or peptide, or an antigen. The mRNAs described herein may be unprocessed or have undergone processing to add a poly(A) tail, be edited in vivo, or have a 5′ cap added. The mRNA molecules may comprise a 5′ UTR or a 3′UTR. The present compositions are contemplated in the delivery of a variety of different mRNA including those which have not undergone processing or have been further processed. Additionally, these nucleic acids may be used therapeutically, used to produce an antibody in vivo, or in a vaccine formulation. mRNA molecules can provide a more direct method of expressing a polypeptide of interest in a target cell. However, such molecules are typically highly liable and rapidly degraded. In some aspects, LNP processing according to the embodiments can be used to substantially stabilize mRNA. In preferred aspects, mRNA is provided encapsulated in or in complex with LNPs.

As mentioned above, in some aspects a nucleic acid molecule of the embodiments encodes a therapeutic polypeptide. For example, the therapeutic protein may be a protein, such as an enzyme that is non-functional or disrupted in a particular disease state (e.g., CFTR in cystic fibrosis).

In further aspects, a polynucleotide of the embodiments encodes an antigen, such as an antigen from a pathogen or a cancer cell-associated antigen. For example, the cancer associated antigen can be CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, GD2, CD123, CD33, CD138, CD23, CD30, CD56, c-Met, mesothelin, GD3, HERV-K, IL-11Ralpha, kappa chain, lambda chain, CSPG4, ERBB2, EGFRvIII or VEGFR2. In some specific aspects the antigen is GP240, 5T4, HER1, CD-33, CD-38, VEGFR-1, VEGFR-2, CEA, FGFR3, IGFBP2, IGF-1R, BAFF-R, TACI, APRIL, Fn14, ERBB2 or ERBB3

Antigens useful in the present disclosure may include those derived from viruses including, but not limited to, those from the family Arenaviridae (e.g., Lymphocytic choriomeningitis virus), Arterivirus (e.g., Equine arteritis virus), Astroviridae (Human astrovirus 1), Birnaviridae (e.g., Infectious pancreatic necrosis virus, Infectious bursal disease virus), Bunyaviridae (e.g., California encephalitis virus Group), Caliciviridae (e.g., Caliciviruses), Coronaviridae (e.g., Human coronaviruses 299E and OC43), Deltavirus (e.g., Hepatitis delta virus), Filoviridae (e.g., Marburg virus, Ebola virus), Flaviviridae (e.g., Yellow fever virus group, Hepatitis C virus), Hepadnaviridae (e.g., Hepatitis B virus), Herpesviridae (e.g., Epstein-Bar virus, Simplexvirus, Varicellovirus, Cytomegalovirus, Roseolovirus, Lymphocryptovirus, Rhadinovirus), Orthomyxoviridae (e.g., Influenzavirus A, B, and C), Papovaviridae (e.g., Papillomavirus), Paramyxoviridae (e.g., Paramyxovirus such as human parainfluenza virus 1, Morbillivirus such as Measles virus, Rubulavirus such as Mumps virus, Pneumovirus such as Human respiratory syncytial virus), Picornaviridae (e.g., Rhinovirus such as Human rhinovirus 1A, Hepatovirus such Human hepatitis A virus, Human poliovirus, Cardiovirus such as Encephalomyocarditis virus, Aphthovirus such as Foot-and-mouth disease virus O, Coxsackie virus), Poxyiridae (e.g., Orthopoxvirus such as Variola virus or monkey poxvirus), Reoviridae (e.g., Rotavirus such as Groups A-F rotaviruses), Retroviridae (Primate lentivirus group such as human immunodeficiency virus 1 and 2), Rhabdoviridae (e.g., rabies virus), Togaviridae (e.g., Rubivirus such as Rubella virus), Human T-cell leukemia virus, Murine leukemia virus, Vesicular stomatitis virus, Wart virus, Blue tongue virus, Sendai virus, Feline leukemia virus, Simian virus 40, Mouse mammary tumor virus, Dengue virus, HIV-1 and HIV-2, West Nile, H1N1, SARS, 1918 Influenza, Tick-borne encephalitis virus complex (Absettarov, Hanzalova, Hypr), Russian Spring-Summer encephalitis virus, Congo-Crimean Hemorrhagic Fever virus, Junin Virus, Kumlinge Virus, Marburg Virus, Machupo Virus, Kyasanur Forest Disease Virus, Lassa Virus, Omsk Hemorrhagic Fever Virus, FIV, SIV, Herpes simplex 1 and 2, Herpes Zoster, Human parvovirus (B19), Respiratory syncytial virus, Pox viruses (all types and serotypes), Coltivirus, Reoviruses-all types, and/or Rubivirus (rubella).

Antigens useful in the present disclosure may include those derived from bacteria including, but not limited to, Streptococcus agalactiae, Legionella pneumophilia, Streptococcus pyogenes, Escherichia coli, Neisseria gonorrhosae, Neisseria meningitidis, Pneumococcus, Hemophilis influenzae B, Treponema pallidum, Lyme disease spirochetes, Pseudomonas aeruginosa, Mycobacterium leprae, Brucella abortus, Mycobacterium tuberculosis, Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Candida albicans, Cryptococcus neoformans, Histoplasma capsulatum, Coccidioides immitis, Blastomyces dermatitidis, Aspergillus fumigatus, Penicillium marneffei, Bacillus anthracis, Bartonella, Bordetella pertussis, Brucella—all serotypes, Chlamydia trachomatis, Chlamydia pneumoniae, Clostridium botulinum—anything from clostridium serotypes, Haemophilus influenzae, Helicobacter pylori, Klebsiella—all serotypes, Legionella—all serotypes, Listeria, Mycobacterium—all serotypes, Mycoplasma-human and animal serotypes, Rickettsia—all serotypes, Shigella—all serotypes, Staphylococcus aureus, Streptococcus—S. pneumoniae, S. pyogenes, Vibrio cholera, Yersinia enterocolitica, and/or Yersinia pestis.

Antigens useful in the present disclosure may include those derived from parasites including, but not limited to, Ancylostomahuman hookworms, Leishmania—all strains, Microsporidium, Necator human hookworms, Onchocerca filarial worms, Plasmodium—all human strains and simian species, Toxoplasma—all strains, Trypanosoma—all serotypes, and/or Wuchereria bancrofti filarial worms.

siRNA

As mentioned above, the present disclosure contemplates the use of one or more inhibitory nucleic acid for reducing expression and/or activation of a gene or gene product. Examples of an inhibitory nucleic acid include but are not limited to molecules targeted to an nucleic acid sequence, such as an siRNA (small interfering RNA), short hairpin RNA (shRNA), double-stranded RNA, an antisense oligonucleotide, a ribozyme and molecules targeted to a gene or gene product such as an aptamer.

An inhibitory nucleic acid may inhibit the transcription of a gene or prevent the translation of the gene transcript in a cell. An inhibitory nucleic acid may be from 16 to 1000 nucleotides long, and in certain embodiments from 18 to 100 nucleotides long.

Inhibitory nucleic acids are well known in the art. For example, siRNA, shRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Since the discovery of RNAi by Fire and colleagues in 1998, the biochemical mechanisms have been rapidly characterized. Double stranded RNA (dsRNA) is cleaved by Dicer, which is an RNAase III family ribonuclease. This process yields siRNAs of ~21 nucleotides in length. These siRNAs are incorporated into a multiprotein RNA-induced silencing complex (RISC) that is guided to target mRNA. RISC cleaves the target mRNA in the middle of the complementary region. In mammalian cells, the related microRNAs (miRNAs) are found that are short RNA fragments (~22 nucleotides). miRNAs are generated after Dicer-mediated cleavage of longer (~70 nucleotide) precursors with imperfect hairpin RNA structures. The miRNA is incorporated into a miRNA-protein complex (miRNP), which leads to translational repression of target mRNA.

In designing a nucleic acid capable of generating an RNAi effect, there are several factors that need to be considered such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Particularly the siRNA exhibits greater than 80, 85, 90, 95, 98% or even 100% identity between the sequence of the siRNA and a portion of a EphA nucleotide sequence. Sequences less than about 80% identical to the target gene are substantially less effective. Thus, the greater identity between the siRNA and the gene to be inhibited, the less likely expression of unrelated genes will be affected.

In addition, the size of the siRNA is an important consideration. In some embodiments, the present disclosure relates to siRNA molecules that include at least about 19-25 nucleotides, and are able to modulate gene expression. In the context of the present disclosure, the siRNA is particularly less than 500, 200, 100, 50, 25, or 20 nucleotides in length. In some embodiments, the siRNA is from about 25 nucleotides to about 35 nucleotides or from about 19 nucleotides to about 25 nucleotides in length.

To improve the effectiveness of siRNA-mediated gene silencing, guidelines for selection of target sites on mRNA have been developed for optimal design of siRNA (Soutschek et al., 2004; Wadhwa et al., 2004). These strategies may allow for rational approaches for selecting siRNA sequences to achieve maximal gene knockdown. To facilitate the entry of siRNA into cells and tissues, a variety of vectors including plasmids and viral vectors such as adenovirus, lentivirus, and retrovirus have been used (Wadhwa et al., 2004).

Within an inhibitory nucleic acid, the components of a nucleic acid need not be of the same type or homogenous throughout (e.g., an inhibitory nucleic acid may comprise a nucleotide and a nucleic acid or nucleotide analog). Typically, an inhibitory nucleic acid form a double-stranded structure; the double-stranded structure may result from two separate nucleic acids that are partially or completely complementary. In certain embodiments of the present disclosure, the inhibitory nucleic acid may comprise only a single nucleic acid (polynucleotide) or nucleic acid analog and form a double-stranded structure by complementing with itself (e.g., forming a hairpin loop). The double-stranded structure of the inhibitory nucleic acid may comprise 16-500 or more contiguous nucleobases, including all ranges derivable thereof. The inhibitory nucleic acid may comprise 17 to 35 contiguous nucleobases, more particularly 18 to 30 contiguous nucleobases, more particularly 19 to 25 nucleobases, more particularly 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

siRNA can be obtained from commercial sources, natural sources, or can be synthesized using any of a number of techniques well-known to those of ordinary skill in the art. For example, commercial sources of predesigned siRNA include Invitrogen's Stealth™ Select technology (Carlsbad, CA), Ambion® (Austin, TX), and Qiagen® (Valencia, CA). An inhibitory nucleic acid that can be applied in the compositions and methods of the present disclosure may be any nucleic acid sequence that has been found by any source to be a validated downregulator of the gene or gene product.

In some embodiments, the disclosure features an isolated siRNA molecule of at least 19 nucleotides, having at least one strand that is substantially complementary to at least ten but no more than thirty consecutive nucleotides of a nucleic acid that encodes a gene, and that reduces the expression of a gene or gene product. In one embodiments of the present disclosure, the siRNA molecule has at least one strand that is substantially complementary to at least ten but no more than thirty consecutive nucleotides of the mRNA that encodes a gene or a gene product.

In one embodiments, the siRNA molecule is at least 75, 80, 85, or 90% homologous, particularly at least 95%, 99%, or 100% similar or identical, or any percentages in between the foregoing (e.g., the disclosure contemplates 75% and greater, 80% and greater, 85% and greater, and so on, and said ranges are intended to include all whole numbers in between), to at least 10 contiguous nucleotides of any of the nucleic acid sequences encoding a target therapeutic protein.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In certain aspects, the RNA molecule contains a 3′-hydroxyl group. Nucleotides in the RNA molecules of the present disclosure can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Publication 2004/0019001 and U.S. Pat. No. 6,673,611 (each of which is incorporated by reference in its entirety). Collectively, all such altered nucleic acids or RNAs described above are referred to as modified siRNAs.

In one embodiment, siRNA is capable of decreasing the expression of a particular genetic product by at least 10%, at least 20%, at least 30%, or at least 40%, at least 50%, at least 60%, or at least 70%, at least 75%, at least 80%, at least 90%, at least 95% or more or any ranges in between the foregoing.

### III. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “−” means a single bond, “═” means a double bond, and “≡” means triple bond. The symbol “----” represents an optional bond, which if present is either single or double. The symbol “ [symbol] =” represents a single bond or a double bond. Thus, for example, the formula

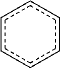

includes

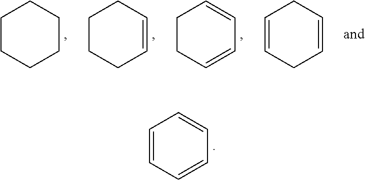

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond

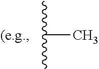

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

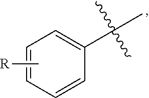

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

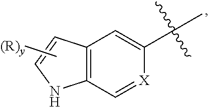

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu orⁱBu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

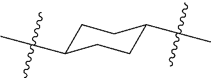 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups-CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

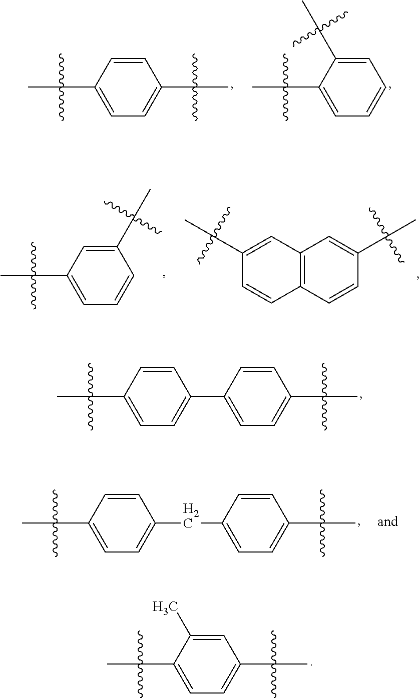

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group-alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

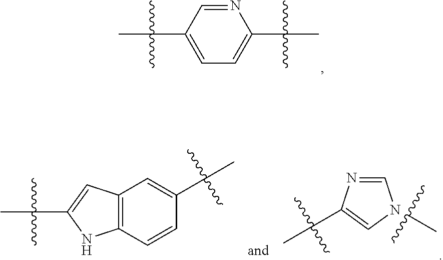

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to an divalent cyclic group, with two carbon atoms, two nitrogen atoms, or one carbon atom and one nitrogen atom as the two points of attachment, said atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

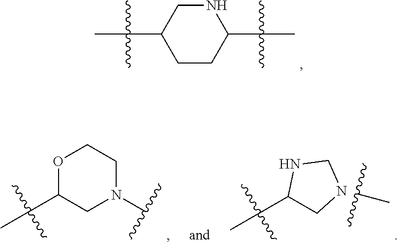

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O) C₆H₅, —C(O) C₆H₄CH₃, —C(O)CH₂C₆H₅, —C(O) (imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group-O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃) (CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(0)₂OH, or —S(O)₂NH₂. The groups —NHC(O)OCH₃ and —NHC(O)NHCH₃ are non-limiting examples of substituted amido groups.

All the compounds of the present invention may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2 ethanedisulfonic acid, 2 hydroxyethanesulfonic acid, 2 naphthalenesulfonic acid, 3 phenylpropionic acid, 4,4′ methylenebis (3 hydroxy 2 ene-1 carboxylic acid), 4 methylbicyclo[2.2.2]oct 2 ene-1 carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o (4 hydroxybenzoyl)benzoic acid, oxalic acid, p chlorobenzenesulfonic acid, phenyl-substituted alkanoic acids, propionic acid, p toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The use of the term “or” in the claims is used to mean “and/or” unless explicitly indicated to refer to alternatives only or the alternatives are mutually exclusive. As used herein “another” may mean at least a second or more.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to +10% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “more substantially free of” or “more substantially free” is used to represent that the composition contains less than 1% of the specific component. The term “essentially free of” or “essentially free” contains less than 0.5% of the specific component.

As used herein, the term “nanoparticle” has its customary and ordinary definition and refers to discrete particles which behave as a whole unit rather than as individual molecules within the particle. A nanoparticle may have a size from about 1 to about 10,000 nm with ultrafine nanoparticles having a size from 1 nm to 100 nm, fine particles having a size from 100 nm to 2,500 nm, and coarse particles having a size from 2,500 nm to 10,000 nm. In some embodiments, the nanoaggregates described herein may comprise a composition of multiple nanoparticles and have a size from about 10 nm to about 100 μm.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

### IV. Examples

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

Provided herein, in certain embodiments, are lipid nanoparticle formulations which may vary in the ratio of the components. The components that may vary in the lipid nanoparticle formulation may independently comprise a cationic ionizable lipid, a helper lipid such as a phospholipid, a PEG lipid, or a sterol such as cholesterol. As disclosed herein, the lipid nanoparticle formulations or pharmaceutical compositions may have different transfection efficiencies depending on the ratios of the components. In some embodiments, the lengths of the alkyl chains present in the cationic ionizable lipids have been altered to improve favorable characteristics or properties of the compositions, such as biodegradability or pulmonary delivery. Provided herein are compositions which show increased mRNA delivery in an air-liquid interface human lung cell model and in healthy mice lungs as well as high distribution at clinically relevant locations in the human lung via next generation impactor experiments. These details and more are described below.

### Example 1—Stably Aerosolized mRNA Lipid Nanoparticles for Lung Delivery

### A. Materials and Methods

In Vitro Transcription (IVT). NLuc mRNA was codon-optimized using the GenSmart™ Codon Optimization online tool through GenScript. Then, a template for NLuc mRNA was created by PCR-amplifying a custom gene block ordered from Twist Biosciences encoding sequences for a T7 promoter, 5′ UTR, codon-optimized NLuc, and 3′ UTR65. All mRNA was synthesized and purified as described previously21,66. Briefly, the amplicon was transcribed using AmpliScribe™ T7-Flash Transcription Kit (Lucigen, ASF-3507) following manufacturer instructions. Following transcription, mRNA was purified using RNA Clean & Concentrator-100 (Zymo, R1019). After purification, the cap1 structure was added using the Vaccinia Capping System (NEB, M2080S) and mRNA Cap 2′-O-methyltransferase (NEB, M0366S). A 3′-poly(A) tail approximately 100 bp long was then added enzymatically using E. coli Poly (A) Polymerase (NEB, M0276L). After polyadenylation, mRNA was purified again using RNA Clean & Concentrator-100. mRNA concentration was determined by Nanodrop 1000 (Thermo Fisher Scientific Inc.), and aliquots were stored at −80° C. until use.

Lipids. The ionizable lipid MC3 ((6Z,9Z,28Z,31Z)-heptatriacont-6,9,28,31-tetraene-19-yl 4-(dimethylamino)butanoate) was purchased from BioFine International Inc. The lipids SM-102 (Heptadecan-9-yl 8-{(2-hydroxyethyl) [6-oxo-6-(undecyloxy) hexyl]amino}octanoate), C-1 (BP-26399), C-2 (BP-26367), C-3 (BP-26361), C-4 (BP-26371), ALC-0135 ([(4-Hydroxybutyl)azanediyl]di(hexane-6,1-diyl) bis(2-hexyldecanoate)), and ALC-0159 (2-[(polyethylene glycol)-2000]-N,N-ditetradecylacetamide) were purchased from BroadPharm. The helper lipids DPPC (1,2-dipalmitoyl-sn-glycero-3-phosphocholine) and DSPC (1,2-distearoyl-sn-glycero-3-phosphocholine) were purchased from Avanti Polar Lipids. The PEG-lipids DMG-PEG 2000 (1,2-dimyristoyl-rac-glycero-3-methoxypolyethylene glycol-2000) and DMPE-PEG 2000 (1,2-dimyristoyl-sn-glycero-3-phosphoethanolamine-N-[methoxy (polyethylene glycol)-2000]) were purchased from NOF America Corporation. Cholesterol was purchased from Sigma-Aldrich.

LNP Formulation. LNPs were formulated using a microfluidic setup described previously (Zhang et al. 2020). Briefly, aliquots of each lipid at 10 mg/mL in 100% ethanol were combined at the appropriate molar ratios. The NLuc mRNA was diluted in 50 mM sodium citrate buffer with pH=3.0. The organic and aqueous phase were combined at a 3:1 ratio with a total flow rate of 9 mL per minute using a NanoAssemblr microfluidic mixer (Precision Nanosystems). NLuc LNPs were formulated at an mRNA concentration of 15 ng/μL for in vitro and in vivo experiments and 100 ng/μL for TEM and NGI experiments with an N/P ratio of 5.67. Cre B-1 was formulated at a concentration of 150 ng/μL with an N/P ratio of 5.67. After formulation, LNPs were dialyzed against 1×PBS pH=7.4 with >500× sample volume overnight using Slide-A-Lyzer™ G2 Dialysis Cassettes with a 10 kDa molecular weight cut-off (87730, Thermo Fisher). Formulations were stored at 4° C. until use.

Aerosol Generation. Each formulation was aerosolized using an Aerogen Solo vibrating mesh nebulizer (Aerogen Ltd.). After aerosol generation, formulations were collected in a 1.5 mL Eppendorf tube for use.

LNP Characterization. Size, polydispersity (PDI), and zeta potential of LNPs were measured using Zetasizer Nano-ZS (Malvern Instruments). All samples were diluted with 0.1×PBS to a final mRNA concentration of 0.75 ng/μL. Size and PDI were measured in a UV-Cuvette micro (759200, BrandTech) and zeta potential was measured in a Folded Capillary Zeta Cell (DTS1070, Malvern).

Encapsulation efficiency was evaluated by the Quant-IT RiboGreen RNA Assay Kit (R11490, Thermo Fisher). LNP samples were prepared to reach a final concentration of 0.6 ng/μL in 1× Tris-EDTA (TE) to measure unencapsulated mRNA and 1% Triton to measure total mRNA. The high-range standard curve was prepared with ribosomal RNA from the kit in both 1× TE and 1% Triton. Both samples and standards were added at a volume of 100 μL to a 96-well black clear bottom plate (3631, Corning). After a 10-minute incubation at 37° C., 100 μL of RiboGreen RNA Reagent diluted 200-fold was added to each well. Sample fluorescence was then measured with a SpectraMax M3 plate reader (Molecular Devices) at 480 nm excitation and 520 nm emission. Encapsulation efficiency was calculated with the following equation: (1-[unencapsulated mRNA]/[total mRNA])*100.

Air-liquid Interface (ALI) Cell Culture. Calu-3 cells (HTB-55, American Type Culture Collection) from passages 12-15 were grown in Eagle's Minimum Essential Media supplemented with 10% fetal bovine serum, 0.1 mM nonessential amino acids, 1 mM sodium pyruvate, and 1× penicillin/streptomycin. After reaching ~80% confluency in a flask, cells were passaged and seeded at a density of 300,000 cells/cm² in 200 μL on the apical side of ThinCert™ CellCoat™ 24 Well Cell Culture Inserts (662641, Greiner Bio-One). After 3 days, media on the apical side was removed and the cells were cultured at ALI. Cells were incubated at 37° C. in a 5% CO₂ atmosphere, and media on the basolateral side was refreshed every 2-3 days.

TEER values were measured using a Millicell ERS-2 with silver/silver chloride electrodes (MERS00002, Millipore Sigma). Transwells were incubated in HBSS on both the apical and basolateral sides for 15 minutes at 37° C. in a 5% CO₂ atmosphere before TEER measurements were taken. Values were calculated by subtracting the resistance of the blank Transwell and then multiplying by the surface area. Cells were used for experiments after one week of ALI culture with TEER values>400 Ω*cm².

In vitro Transfection Efficiency. Transfection efficiency of LNPs were assessed in ALI Calu-3 cells. LNPs were added to the apical side of the ALI Calu-3 cells in 100 μL final volume. Cells were harvested 24 hours later by scraping each transwell and transferring with a multichannel to a white plate containing with 100 μL of furimazine from the Nano-Glo® Luciferase Assay System (N1110, Promega). Samples were incubated for 3 minutes at room temperature before analysis with a SpectraMax M3 plate reader (Molecular Devices).

In vivo transfection efficiency with Balb/c mice. Animal protocols were approved by the Institutional Animal Care and Use Committee (IACUC) at the University of Texas at Austin. Balb/c mice (female, 6-8 weeks) were purchased from Charles River. Mice were acclimated for at least one week before the study.

Mice were dosed intratracheally with 750 ng of LNP in approximately 50 μL volume while anesthetized under a continuous flow of 2% isoflurane. After 24 hours, mice were sacrificed, and the lungs were harvested and separated into the five lobes (left, cranial, accessory, middle, and caudal). Lobes were submerged in 400 μL of substrate from the Nano-Glo® Luciferase Assay System for 5 minutes. Luminescence was measured with In Vivo Imaging System (IVIS) with an exposure time of 1 second, medium binning, and an F-stop of 1 and quantified with Living Image (PerkinElmer).

In vivo delivery with Ai9 mice. Ai9 mice (female, 6-8 weeks) were purchased from Jackson (007909). Mice were acclimated for at least one week before the study. Mice were dosed intratracheally with LNPs delivering 0.5 mg/kg Cre mRNA (L-7211, TriLink) in approximately 50 μL while anesthetized under a continuous flow of 2% isoflurane. Mice were dosed once every other day over a period of 4 days. Three days after the last dose, the lungs were harvested.

To visualize the fluorescence of tdTomato, the lungs were imaged with In Vivo Imaging System (IVIS) at an exposure time of 5 seconds with 535 nm excitation and 580 nm emission, medium binning, and an F-stop of 1.

To prepare the lung cells for single cell suspension, the tissue was finely minced and incubated for 1 hours at 37° C. in digestion medium consisting of 90 units/mL Collagenase Type I (SCR103, Millipore Sigma), 50 units/mL DNase I (11284932001, Sigma-Aldrich), and 60 units/mL Hyalurinodase (H3506, Sigma-Aldrich) in DMEM as previously described (Cheng et al., 2020). After incubation, the digestion medium was quenched with DMEM+20% FBS, and tissues were filtered through a 70 μm filter. Cells were washed once with 1×PBS before ACK lysis with 5 mL ACK Lysing Buffer (A1049201, ThermoFisher) for 3 minutes at room temperature. Cells were washed in 1×PBS and resuspended in 50 μL 1×PBS for staining.

Each sample was stained with 1 μL of 1 1:10 dilution of Zombie NIR (423105, BioLegend) and incubated at 4° C. for 30 minutes. Cells were then washed with 1 mL of 1×PBS twice, resuspended in 50 μL 1×PBS+2% FBS+0.05% sodium azide, and then stained with 1 μL of each of the following antibodies: Pacific Blue anti-mouse CD45 (103126, BioLegend), Alexa Fluor 488 anti-mouse CD31 (102414, BioLegend), and Alexa Fluor 647 anti-mouse CD326 (Ep-CAM) (118212, BioLegend). Cells were analyzed using the Attune NxT Flow Cytometer.

Next Generation Impactor (NGI). Aerosol distributions of LNPs were assessed through NGI (MSP Corporation). A T-piece plug (device) was used to connect the Aerogen Solo vibrating mesh nebulizer to the induction port. Before each run, all NGI components were transferred to a cold room to prechill to 4° C. for at least 90 minutes (Berg et al., 2007). LNPs were loaded into the nebulizer at 1 mL per formulation. The flow rate was operated at 15 L/min. The cut-off diameters for each stage at this flow rate are 14.1 μm for stage 1, 8.61 μm for stage 2, 5.39 μm for stage 3, 3.30 μm for stage 4, 2.08 μm for stage 5, 1.36 μm for stage 6, and 0.98 μm for stage 758,69.

The LNPs were collected by first wrapping the induction port and device with parafilm. Then, 3 mL of 1×TE was added to these components as well as stages 1-7 and the MOC. The buffer was swirled around each component ~10 times. After collection, the amount of mRNA in each stage was determined with the RiboGreen assay.

Calculation of EF, MMAD, GSD, and FPF_{5 μm}. The cut-off diameters used for each stage at a flow rate of 15 L/min were 14.1 μm for stage 1, 8.61 μm for stage 2, 5.39 μm for stage 3, 3.30 μm for stage 4, 2.08 μm for stage 5, 1.36 μm for stage 6, and 0.98 μm for stage 7. The concentration calculated according to the method above was used to determine the aerodynamic diameters of stages 1-7 using Equation 1 (Patel et al., 2020; Yanez Arteta et al., 2018).

$${D}_{50 , Q} = A * ( \frac{15}{Q} ) {B}^{*} \qquad ( 1 )$$

In this equation, Q is the flow rate through the impactor, D_{50,Q} is the aerodynamic diameter at the flow rate, Q, and A* and B* are empirical constants determined for Q=15 L/min for use with flow rates between 15-30 L/min. The EF was calculated by dividing the mass deposited in the throat, stages 1-7, and the MOC by the mass deposited in all components of the NGI. FPF_{5 μm} was interpolated from the graph of cumulative percentage of emitted dose plotted versus particle cutoff size. The MMAD was calculated by plotting the log cumulative fraction of drug versus the aerodynamic diameter. The GSD was calculated with the aerodynamic diameters corresponding to the 15.87% and the 84.13% determined by plotting the cumulative percentage of mass less than the stated aerodynamic diameter versus aerodynamic diameter (log).

Transmission Electron Microscopy (TEM). LNP structures were visualized using a FEI Tecnai TEM. For each sample, 5 μL of LNP were loaded onto a Carbon Type-A 300 mesh Copper grid (01820, Ted Pella). After incubation for 5 minutes, the sample was blotted off with filter paper, and the grid was washed with 5 μL of H₂O. Negative staining was performed by adding 5 μL of 2% uranyl acetate. Imaging was performed at 43,000× magnification while operating at 80 kV.

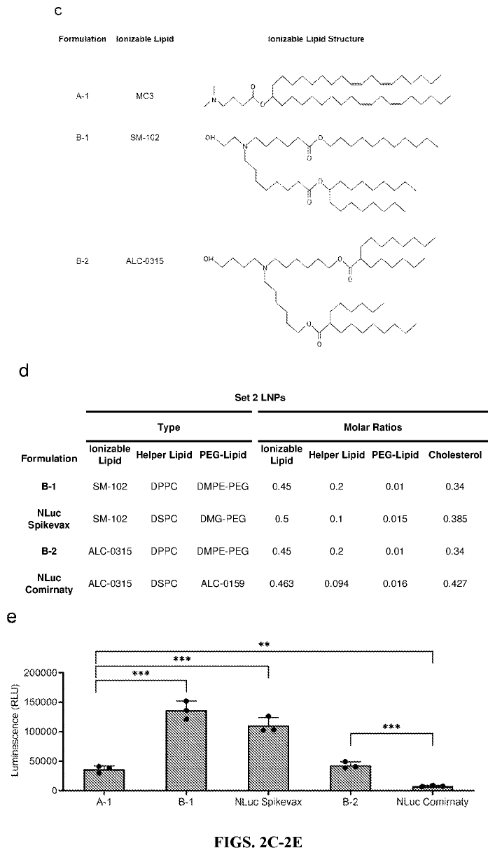

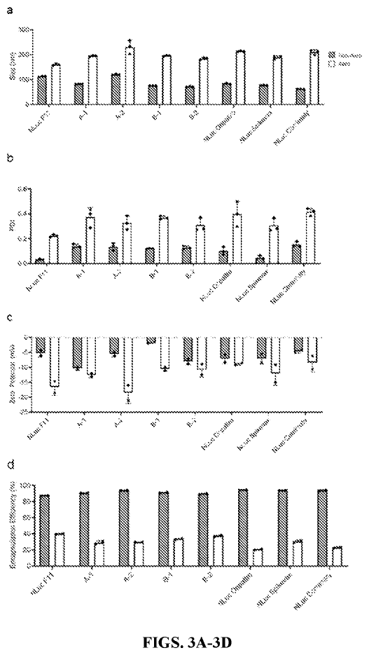

Statistical analysis. All in vitro experiments were completed with three replicates while all in vivo experiments were completed with four replicates. All P values were calculated using a student's t-test. The *p-values<0.05, **p-values<0.01, ***p-values<0.001, and ****p-values<0.0001 were considered statistically significant. Data values were presented as mean±standard deviation. All analysis was done in GraphPad Prism (Version 8.4.3). For FIGS. 2 and 3, a simple linear regression was used to determine statistical significance.

### B. Results and Discussion

### (1) Results.

Screening Aerosolized LNPs Enhances Transfection Efficiency in Air-liquid Interface (ALI) lung cells. The effect of varying LNP component ratios based on a known formulation F11 (Zhang et al., 2020b) was evaluated. This LNP had a base composition of MC3, DPPC, DMPE-PEG, and cholesterol (0.6/0.2/0.01/0.19) and was a lead candidate in delivering eGFP mRNA to plated lung cells and Fluc mRNA to Balb/c mice lungs. From F11, a first set of 2 LNPs with varying MC3, DPPC, and cholesterol component ratios to a constant amount of PEG-lipid (0.01 molar ratio) (FIG. 2A) was developed. The mRNA LNP delivery for the set of developed LNPs was compared to F11 and Onpattro, a clinically approved siRNA LNP also using MC3. All formulations encapsulated NanoLuc mRNA, a reporter molecule that is 100× more sensitive than traditional firefly luciferase. Compositions of comparison LNPs F11 and Onpattro which comprise encapsulated NanoLuc mRNA are also termed herein “NLuc F11” and “NLuc Onpattro”, respectively.

The transfection efficiency was subsequently assessed by delivering 1000 ng of each aerosolized LNP to Calu-3 cells cultured at ALI, a physiologically relevant cell culture model for the human lungs. At ALI, Calu-3 cells produce mucus and form tight functions making them a good model for screening formulations for inhaled delivery as they acquire in vivo-like barrier properties such as formation of tight junctions and secretion of mucus components (Grainger et al., 2006; Fiegel et al., 2003; Bivas-Benita et al., 2004; Amidi et al., 2006; Grenha et al., 2007). To establish ALI, Calu-3 cells were exposed to air on the apical side of the transwell while maintaining contact with media on the basolateral side. To quantify tight junction integrity, transepithelial electrical resistance (TEER) was measured. After being cultured at ALI for one week, Calu-3 cells demonstrated intact tight junctions with TEER values>400 Ω*cm². After aerosolization, 1000 ng of each LNP was delivered to the apical side of the transwell and incubated for 24 hours. Twenty-four hours post administration, the cells were harvested and luminescence intensity was measured. Decreasing the MC3 ratio from 0.6 to 0.45 resulted in significantly higher luminescence for both A-1 and A-2 compared to NLuc F11 (FIG. 2B). The best performing formulation, A-1, had a greater than 2.6-fold increase compared to NLuc F11, and a 1.3-fold increase compared to NLuc Onpattro. As A-1 had the highest luminescence, it was taken for further studies.

It was thought that, without being bound by theory, that altering the type of cationic ionizable lipid could increase the potency. To date, there have been two other approved cationic ionizable lipids besides MC3: SM-102 for the Moderna mRNA LNP COVID-19 vaccine and ALC-0315 for the Pfizer/BioNTech COVID-19 vaccine. While A-1 is an MC3 based LNP, its component ratios and component types are similar to these vaccines (Baden et al., 2020; Polack et al., 2020). Therefore, a second set of 2 LNPs was formulated which comprised the cationic ionizable lipid of SM-102 (B-1) and ALC-0315 (B-2) with component ratios, helper lipid type, PEG-lipid type, and cholesterol type constant as provided by composition A-1 (FIG. 2C & FIG. 2D).

The COVID-19 vaccines Spikevax and Comirnaty were formulated to serve as comparison LNPs for B-1 and B-2, respectively. As these LNPs encapsulated NLuc rather than the full-length SARS-COV-2 spike protein, the comparison formulations are referred to herein as “NLuc Spikevax” and “NLuc Comirnaty” as appropriate. These formulations were screened in the same manner as set 1 LNPs. After aerosolization, B-1 had the highest transfection efficiency, with a greater than 3-fold increase compared to A-1. However, B-2 did not exhibit significantly higher luminescence than A-1 (FIG. 2E). This result was unexpected as SM-102 and ALC-0315 have similar structures and there are major structural differences between SM-102 and ALC-0315 considered together or separately with respect to MC3. More specifically, SM-102 and ALC-0315 both have one hydroxy head group, one tertiary amine, two esters, and saturated hydrocarbons, MC3 has a dimethylamine head group and an unsaturated di-linoleic lipid tail. Without being bound by theory, the structure of the cationic ionizable lipid had a greater impact on transfection efficiency after aerosolization than the component ratios. In addition, both B-1 and B-2 LNP formulations had higher luminescence compared to their vaccine counterparts although this increase was not statistically significant for B-1. Taken together, both screens identify a lead candidate, B-1, while showing that the importance of CILs in enhancing delivery of mRNA LNPs for ALI Calu-3 cells.

To investigate whether an improved transfection before aerosolization correlated with improved transfection after aerosolization, non-aerosolized and aerosolized set 1 and 2 LNPs (FIG. 8) were compared. Particles with lower transfection compared to A-1 before aerosolization had lower transfection after aerosolization. For example, NLuc F11 had decreased luciferase expression both before and after aerosolization compared to A-1.

However, this was not the case for particles with higher transfection. Both B-1 and NLuc Spikevax had significantly higher luciferase expression after aerosolization compared to A-1 despite all three LNPs having similarly high luciferase expression before aerosolization. These results were consistent with previous observations that some formulations have a large decrease in delivery after aerosolization despite being identified as lead candidates before aerosolization (Zhang et al., 2020). Overall, these data show, without being bound by theory, that an improved transfection before aerosolization does not necessarily ensure improved transfection after aerosolization. As can be seen, post-aerosolization transfection efficiency can be unpredictable based on composition or pre-aerosolization transfection efficiency. In addition, the size, polydispersity index, zeta potential, and encapsulation efficiency before and after nebulization had no correlation with transfection efficiency (FIGS. 3A-3D). Importantly, all particles were stable before aerosolization with sizes ranging from 62.38±0.49 to 120.46±1.11 and PDIs below 0.2.

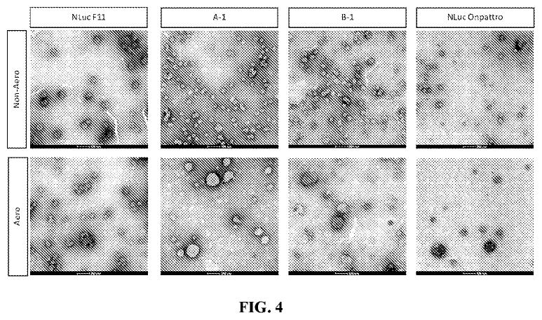

Characterization of LNP morphology before and after aerosolization. TEM was used to evaluate the morphology of A-1 and B-1 before and after aerosolization. This allowed comparison of the effect of the cationic ionizable lipid type (MC3 vs. SM-102) on aerosolization stability. Before aerosolization, A-1 and B-1 had the same spherical structure noted by other TEM images of mRNA LNPs (FIG. 4) (Kim et al., 2021; Kulkarni et al., 2019; Patel et al., 2020; Carrasco et al., 2021; Eygeris et al., 2020). After aerosolization, both had roughly spherical shapes with a larger size distribution as shown in the DLS data (FIG. 3A, FIG. 3B). However, B-1 was observed to have more defects on the surface of the aerosolized LNP as compared to A-1. In other studies of LNPs, though without being bound by the theory, faceted surfaces such as those of B-1 have been associated with more efficient membrane fusion and therefore increased endosomal escape (Patel et al., 2020). Without being bound by theory, the four double-bonds in MC3 may play limit the flexibility of the LNP structure upon aerosolization whereas the less constrained SM-102 structure, with no double bonds, may be more susceptible to structural changes under shear forces. These differences demonstrate, without being bound by theory, the importance of CIL for aerosolized mRNA LNPs and show that the most morphologically intact particle may not have the highest transfection efficiency. In this way, the transfection efficiency of the LNPs disclosed herein is not predictable or discernable from the morphology of the particle.

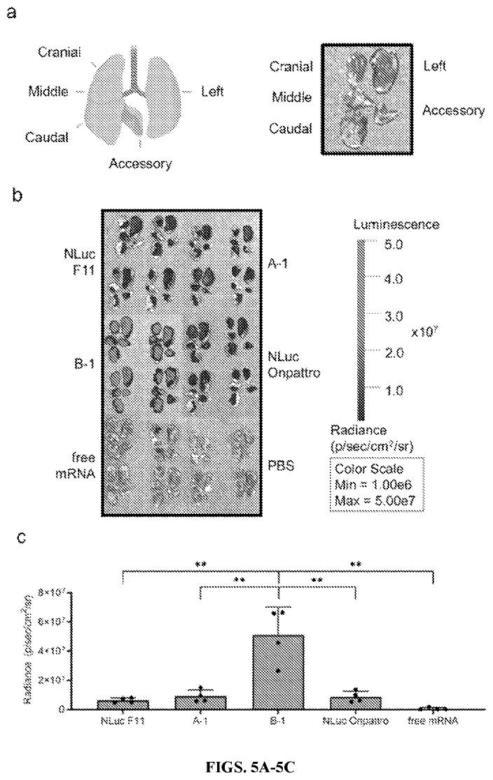

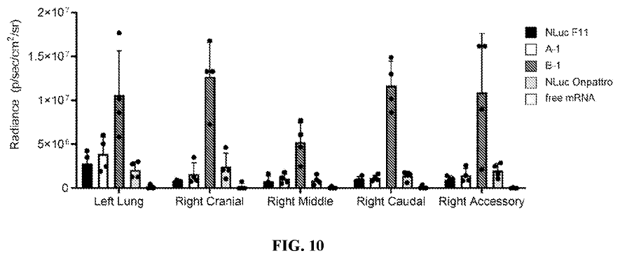

In vivo delivery of mRNA LNPs to mouse lungs. To evaluate the transfection efficiency of the NanoLuc mRNA LNPs in lung epithelial cells, 750 ng each of aerosolized F11, NLuc F11, A-1, B-1, and NLuc Onpattro were delivered separately to Balb/c mice through intratracheal administration after aerosolization by vibrating mesh nebulizer. Unencapsulated NLuc mRNA (free mRNA) and PBS served as negative controls. Mice lungs were harvested and separated into five individual lobes (left, cranial, accessory, middle, and caudal) 24 hours after administration for staining in furimazine (FIG. 5A). All formulations had similar distributions throughout each of the five lobes. Additionally, all LNPs achieved higher mRNA expression compared to free mRNA throughout all lung lobes (FIG. 10). Overall, B-1 exhibited the highest radiance. The transfection efficiency of B-1 was 8.1×, 5.6×, 5.8×, and 103.2× higher than NLuc F11, A-1, NLuc Onpattro, and free mRNA, respectively (FIG. 5B, FIG. 5C). Although A-1 and NLuc Onpattro exhibited increased transfection compared to NLuc F11, the difference was not statistically significant. Therefore, B-1 was selected as the lead candidate from its delivery efficacy in both ALI Calu-3 cells and healthy mouse lungs.

Assessment of cell types transfected by B-1 in the lungs. To track delivery and quantify the ability of B-1 to transfect therapeutically relevant cell types within the lungs after aerosolization, the Ai9 reporter mouse model (FIG. 9A) was utilized (Madisen et al., 2010). These mice contain a stop cassette flanked by LoxP sites in the Rosa26 locus preventing transcription of a fluorescent tdTomato protein. Upon Cre enzyme-mediated recombination of the LoxP sites, the stop cassette will be deleted and tdTomato will be expressed. tdTomato positive cells can subsequently be isolated in conjunction with select surface markers via flow cytometry to identify which cell type the presently studied formulation is transfecting.

B-1 LNP encapsulating Cre mRNA was delivered and the lungs were harvested after seven days for analysis (FIG. 9A). Through fluorescent imaging, B-1 had significantly higher radiance than the PBS control (FIG. 9B). Through flow cytometry, it was found that 8.9% of the epithelial cells, 1.9% of the immune cells, and 0.6% of the endothelial cells were tdTomato positive (FIG. 9C). Upon local delivery, there is greater relative uptake in epithelial cells compared to other cell types, which is important in CF where the epithelia are the primary target for gene editing therapies. It has been shown that approximately 5-50% of epithelial cells need to express functional CFTR to improve lung function in CF patients (Chu et al., 2015); these findings indicate that the cell transfection properties of aerosolized B-1 are promising.

B-1 LNP targets relatively more of the desired epithelial cells compared to other lung-targeted LNPs (Cheng et al., 2020). Compared to an intravenously delivered five-component LNP with tropism for the lung, B-1 has higher relative uptake in epithelial cells compared to immune cells (4.7-fold vs. 1.9-fold). Additionally, the transfection of B-1 in endothelial cells is significantly lower (0.6% vs. 66%). Here, the route of administration can play a role in delivery. The presently disclosed LNP formulation is intended for local, pulmonary delivery to reach the epithelia, whereas the other formulation is delivered intravenously and must overcome the vascular endothelium before it can access the target epithelial cells. For at least this reason, the present invention provides an advantage for localized delivery for LNP uptake in lung epithelial cells.

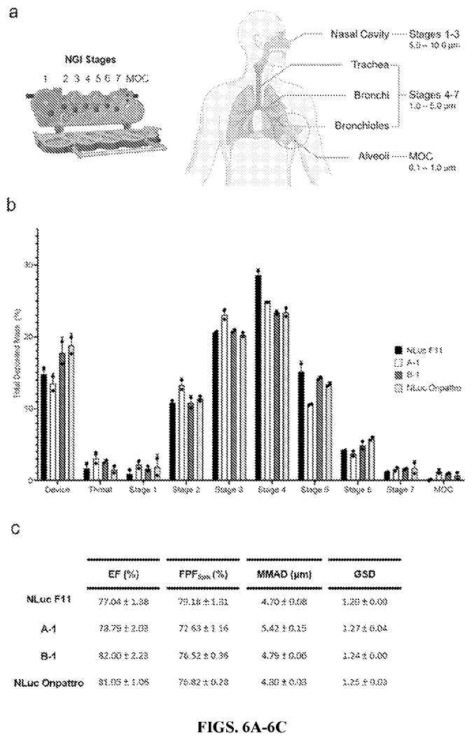

Next generation impaction to evaluate aerodynamic properties. A successful inhaled therapy must have droplet sizes ranging from 1-5 μm to avoid being expired or caught in the upper airways (Chow et al., 2020). To evaluate the aerosol distribution across the human lungs, a Next Generation Impactor (NGI) was used. This device uses an airflow to allow aerosolized formulations to deposit along a throat, seven stages, and a micro-orifice collector (MOC). The airflow rate through the NGI determines the aerodynamic diameter of the particles in each component and therefore their distribution within the lungs. At a flow rate of 15 L/min, particles collected from stages 1-3 deposit in the nasal cavity while particles collected from stages 4-7 and the MOC deposit throughout the therapeutically relevant areas of the lung (FIG. 6A) (Marple et al., 2004). The device was used to measure the aerodynamic diameter and lung deposition of NLuc F11, A-1, B-1, and NLuc Onpattro upon aerosolization (FIG. 6B & FIG. 6C). The flow rate was operated at 15 L/min to mimic tidal breathing, and therefore the cut-off diameters for each stage are 14.1 μm for stage 1, 8.61 μm for stage 2, 5.39 μm for stage 3, 3.30 μm for stage 4, 2.08 μm for stage 5, 1.36 μm for stage 6, and 0.98 μm for stage 7. After each run, LNPs were collected from the NGI components and quantified with the RiboGreen assay. These values were used to calculate the emitted fraction (EF), fine particle fraction <5 μm (FPF_{5 μm}), median mass aerodynamic diameter (MMAD), and geometric standard deviation (GSD) (U.S. Pharmacopeia, 2003). The EF is the percentage of all particles deposited in the NGI components that are recovered from the throat, stages 1-7, and MOC. The FPF_{5 μm} is the percentage of emitted particles that have diameters <5 μm. The MMAD is the diameter at which 50% of the aerosol droplets are larger or smaller with a benchmark value being the limit of settling in the lower airways (5 μm). The GSD represents the spread of the aerosol particle size distribution. A majority of A-1, B-1, and NLuc F11 and NLuc Onpattro controls that deposited within the NGI components were collected from stages 4-7 and the MOC, which indicates the potential for deposition throughout the small and large airways (FIG. 6B). This correlates with the high EFs (77.04%-82.00%) (FIG. 6C). Importantly, the FPF_{5 μm} range of 72.63%-79.18% highlights that a majority of the particles are within the respirable range. In comparison, this fraction is greater than that of Arikayce (FPF_{5 μm}: 50.3-53.5%), the only clinically approved liposomal drug developed for inhalation (Li et al., 2021; Leong and Ge, 2022). Therefore, the present invention represents an improvement over solutions known in the art. Compared to Arikayce, the MMAD is also <5 μm for all LNPs except A-1. In addition, the GSD was also less than 2 for all LNPs.

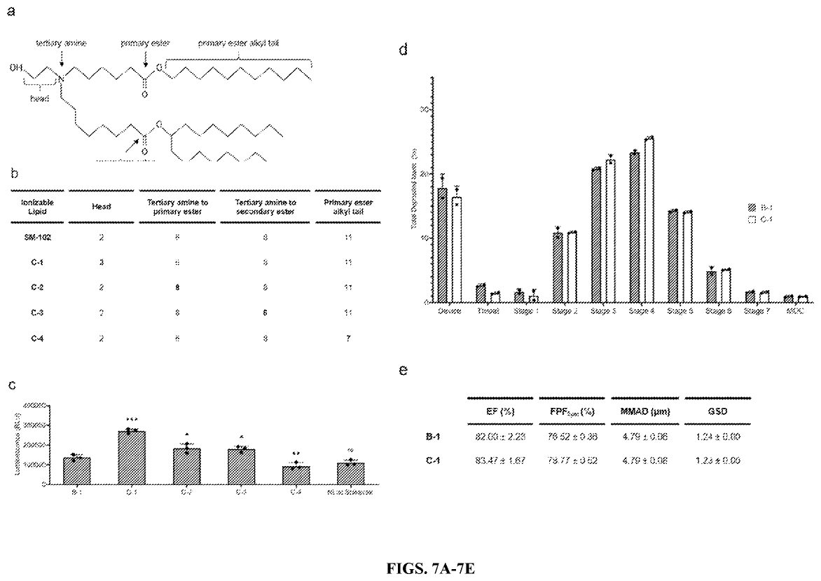

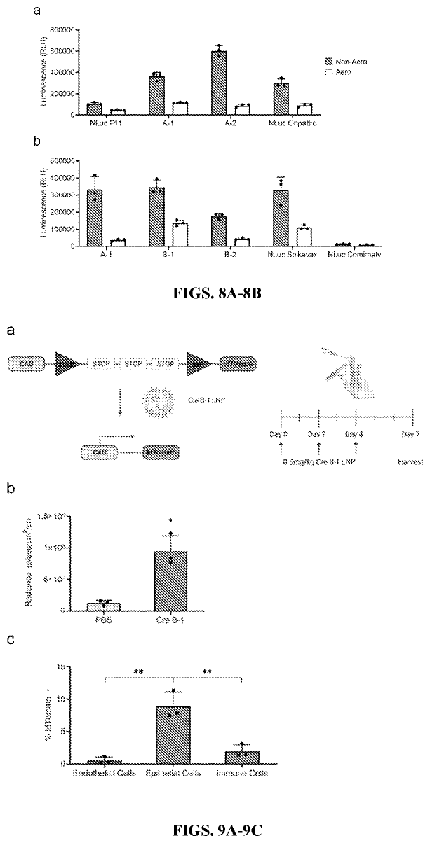

Structure-function analysis on SM-102. While SM-102 is clinically approved for the Moderna COVID-19 vaccine, identifying structural components that improve transfection efficiency after aerosolization is valuable for lung delivery. In a previous study, cationic ionizable lipids with structural similarity to SM-102 had significant effects on transfection efficiency in CD-1 mice (Sabnis et al., 2018). SM-102 structure-function was probed by screening four analogs that differed in the number of carbons in the head, tail, and carbon chain from the primary nitrogen to both esters (FIG. 7A, FIG. 7B). Each of the analogs was formulated into LNPs (C1-C4) and compared to B-1 while maintaining the A-1 component ratios. NLuc Spikevax was formulated as the benchmark. Twenty-four hours after delivering 1000 ng of aerosolized LNPs to ALI Calu-3 cells, the cells were harvested and the luminescence intensity was measured.

Most analogs produced significant effects compared to SM-102 (FIG. 7C). Without being bound by theory, the increased number of carbons in the head structure of C-1 resulted in the highest delivery, with a 2-fold increase in luminescence compared to B-1. Additionally, changing the number of carbons from the amines to the two esters in C-2 and C-3 also increased delivery as supported by a 1.3-fold increase in luminescence for LNPs formed from each. However, the shorter tail of C-4 decreased delivery by 1.5-fold.

Similarly to B-1, a majority of C-1 deposits within stages 4-7 and the MOC (FIG. 7D). Additionally, C-1 has a high EF (83.47±1.67%) and FPF_{5 μm} (78.77±0.52%) as well as an MMAD below <5 μm (4.79±0.08 μm) and a GSD below 2 (1.23±0.00) (FIG. 7E). While the chemistry may improve the transfection efficiency for SM-102 analogs, this does not correspond with any particular impact on the potential for deposition in the human lungs.

### (2) Discussion

The present disclosure provides compositions that have reduced degradation during aerosolization and have improved particle deposition in therapeutically relevant locations. As an example, B-1 was stable upon aerosolization and achieved the highest transfection efficiency in ALI human lung cells and in healthy mice lungs. Further, Cre B-1 had an increased transfection efficiency in epithelial cells (8.9%) compared to immune cells (1.9%) or endothelial cells (0.6%) in the lung. The high transfection rate of aerosolized Cre B-1 in epithelial cells is promising for pulmonary diseases like CF (Marquez et al., 2021). In addition, B-1 also had the desired droplet properties in simulated human airways. Through NGI, B-1 exhibited an FPF_{5 μm} higher than Arikayce, the only clinically approved liposomal drug for inhalation with a mesh nebulizer (Li et al., 2021; Leong and Ge, 2022). Importantly, a high FPF_{5 μm} translates to a greater percentage of therapeutically active mRNA. While transfection experiments and NGI assess different drug characteristics, each method had valuable criteria for an aerosolized formulation. Transfection shows the ability of the cell to translate and express the protein of interest after mRNA delivery while NGI shows the potential of the formulation to deposit in clinically relevant locations throughout simulated human airways. A therapeutically relevant LNP must be able to do both. LNPs of the present disclosure have improved transfection or improved deposition in clinically relevant locations as described elsewhere in the Examples. The present disclosure also provides for more potent LNPs, such as those formulated with 3 carbons in the head structure instead of 2 as illustrated, as one non-limiting example, by analog C-1 above. Therefore, the compositions of the present disclosure represent a solution for new compositions for pulmonary delivery.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Akinc et al., Nature Nanotechnology 14, 1084-1087 (2019).
Amidi et al., J. Control. Release, 111, 107-116 (2006).
Baden et al., N Engl J Med. NEJMoa2035389 (2020).
Berg et al., Journal of Aerosol Medicine. 20, 97-104 (2007).
Bivas-Benita et al., Eur. J. Pharm. Biopharm. 58, 1-6, (2004).
Carrasco et al., Commun Biol. 4, 1-15 (2021).
Chapter 601-Physical tests and determinations: aerosols. In: United States Pharmacopeia: USP31-NF26.; 2003.
Cheng et al., Nature Nanotechnology. 15, 313-320 (2020).
Chow et al., Trends Pharmacol Sci. 41, 715-729 (2020).
Chu et al., Nat. Biotechnol., 33 (5): 543-548, (2015)
Cipolla et al., Pulmonary Drug Delivery. 4, 1047-1072 (2013).
Eygeris et al., Nano Lett. 20, 4543-4549 (2020).
Fiegel et al., Pharm Res, 20, 788-796 (2003).
Garbuzenko et al., Pharm Res. 26, 382-394 (2009).
Grainger et al., Pharm. Res. 23, 1482-1490 (2006).
Grenha et al., Eur J Pharm Sci, 31, 73-84 (2007).
Jayaraman et al., Angewandte Chemie International Edition. 51, 8529-8533 (2012).
Kim et al., Science Advances. 7 eabf4398. doi.org/10.1126/sciadv.abf4398. (2021).
Kim et al., ACS Nano. doi.org/10.1021/acsnano.2c05647. (2022).
Kulkarni et al., ACS Nano. 12, 4787-4795 (2018).
Kulkarni et al., Nanoscale. 11, 9023-9031 (2019).
Leong and Ge, Biomedicines 10 2179 (2022).
Li et al., Eur J. Pharm. Biopharm. 166, 10-18 (2021).
Lokugamage et al., Nat Biomed Eng. 5, 1059-1068 (2021).
Madisen et al., Nat Neurosci, 13, 133-140 (2010).
Marple et al., J Aerosol Med. 17, 335-343 (2004)
Marquez Loza et al., Mol Ther Methods Clin Dev. 21, 94-106 (2021).
Polack et al., N Engl J Med. NEJMoa2034577 (2020).
Patel et al., Nature Communications. 11, 983 (2020).
Qiu et al., Proceedings of the National Academy of Sciences. 118, e2020401118 (2021).
Qiu et al., Proceedings of the National Academy of Sciences. 119, e2116271119 (2022).
Rudokas et al., Med Princ Pract. 25 60-72, (2016).
Sabnis et al., Mol Ther. 26, 1509-1519 (2018).
Translate Bio, Inc., clinicaltrials.gov (2020).
Woo et al., Pulmonary Pharmacology & Therapeutics 75, 102134 (2022).
Yanez Arteta et al., Proceedings of the National Academy of Sciences 115, E3351-E3360 (2018).
Zhang et al., Sci Adv 6, eabc2315 (2020a).
Zhang et al., Pharmaceutics 12, 1042 (2020b).

What is claimed is:

1. A pharmaceutical composition comprising:
a) a lipid nanoparticle (LNP); wherein the LNP comprises:
i) a cationic ionizable lipid (CIL); wherein the cationic ionizable lipid is of the formula: 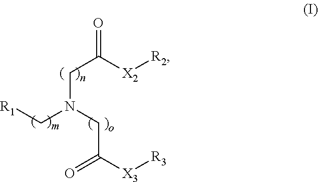
wherein:
m, n, and o are each independently 1, 2, 3, 4, 5, 6, 7, 8, 9, or 10;
X₂ and X₃ are each independently —O— or —N—;
R₁ is hydroxy, amino, halo, or mercapto; or alkoxy_{(C≤8)}, alkylamino_{(C≤8)}, dialkylamino_{(C≤12)}, or a substituted version of any of these groups; and
R₂ and R₃ are each independently alkyl_{(C≤24)}, alkenyl_{(C≤24)}, or alkynyl_{(C≤24)};
or a pharmaceutically available salt thereof;
ii) a phospholipid;
iii) a PEG-lipid; and
iv) a sterol; and
b) a nucleic acid;
wherein the composition is formulated for aerosolization.

2. The pharmaceutical composition of claim 1, wherein the cationic ionizable lipid is further defined as: 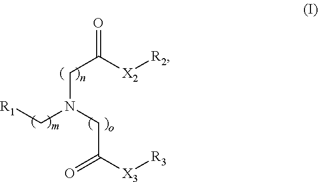
wherein:
m is 1, 2, 3, 4, or 5;
n and o are each independently 1, 2, 3, 4, 5, 6, 7, 8, 9, or 10;
X₂ and X₃ are each independently —O— or —N—;
R₁ is hydroxy, amino, halo, or mercapto; or alkoxy_{(C≤8)}, alkylamino_{(C≤8)}, dialkylamino_{(C≤12)}, or a substituted version of any of these groups; and
R₂ and R₃ are each independently alkyl_{(C≤24)}, alkenyl_{(C≤24)}, or alkynyl_{(C≤24)};
or a pharmaceutically available salt thereof.

3. The pharmaceutical composition of claim 2, wherein the cationic ionizable lipid is further defined as: 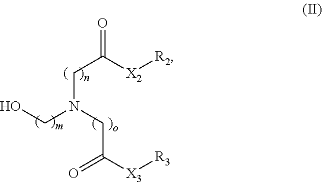
wherein:
mis 1, 2, 3, 4, or 5;
n and o are each independently 1, 2, 3, 4, 5, 6, 7, 8, 9, or 10; and
X₂ and X₃ are each independently —O— or —N—; and
R₂ and R₃ are each independently alkyl_{(C≤24)}, alkenyl_{(C≤24)}, or alkynyl_{(C≤24)};
or a pharmaceutically available salt thereof.

4. The pharmaceutical composition of claim 3, wherein the cationic ionizable lipid is further defined as: 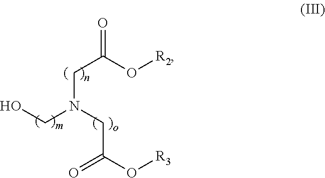
wherein:
m is 1, 2, 3, 4, or 5;
n and o are each independently 1, 2, 3, 4, 5, 6, 7, 8, 9, or 10; and
R₂ and R₃ are each independently alkyl_{(C≤24)}, alkenyl_{(C≤24)}, or alkynyl_{(C≤24)};
or a pharmaceutically available salt thereof.

5. The pharmaceutical composition according to any one of claims 1-4, wherein m is 1, 2, 3, or 4.

6. The pharmaceutical composition of claim 5, wherein m is 2 or 3.

7. The pharmaceutical composition of claim 6, wherein m is 2.

8. The pharmaceutical composition of claim 6, wherein m is 3.

9. The pharmaceutical composition according to any one of claims 1-8, wherein n is 3, 4, 5, 6, 7, 8, or 9.

10. The pharmaceutical composition of claim 9, wherein n is 4, 5, 6, 7, or 8.

11. The pharmaceutical composition of claim 10, wherein n is 5 or 7.

12. The pharmaceutical composition of claim 11, wherein n is 5.

13. The pharmaceutical composition of claim 11, wherein n is 7.

14. The pharmaceutical composition according to any one of claims 1-13, wherein o is 3, 4, 5, 6, 7, 8, or 9.

15. The pharmaceutical composition of claim 14, wherein o is 4, 5, 6, 7, or 8.

16. The pharmaceutical composition of claim 15, wherein o is 5 or 7.

17. The pharmaceutical composition of claim 16, wherein o is 5.

18. The pharmaceutical composition of claim 16, wherein o is 7.

19. The pharmaceutical composition according to any one of claims 1-12 and 14-16 and 18, wherein n is 5 and o is 7.

20. The pharmaceutical composition according to any one of claims 1-12 and 14-17, wherein n and o are each 5.

21. The pharmaceutical composition according to any one of claims 1-11, 13, and 14-17, wherein n is 7 and o is 5.

22. The pharmaceutical composition according to any one of claims 1-11, 13, 14-16, and 18, wherein n and o are each 7.

23. The pharmaceutical composition according to any one of claims 1-22, wherein R₂ is alkyl_{(C≤24)} or substituted alkyl_{(C≤24)}.

24. The pharmaceutical composition according to any one of claims 1-23, wherein R₂ is alkyl_{(C≤24)}.

25. The pharmaceutical composition according to any one of claims 1-24, wherein R₂ is alkyl_{(C6-20)}.

26. The pharmaceutical composition according to any one of claims 1-25, wherein R₂ is n-alkyl_{(C6-20)}.

27. The pharmaceutical composition according to any one of claims 1-26, wherein R₂ is n-alkyl_{(C6-12)}.

28. The pharmaceutical composition according to any one of claims 1-27, wherein R₂ is n-hexane, n-heptane, n-octane, n-nonane, n-decane, n-undecane, or n-dodecane.

29. The pharmaceutical composition according to any one of claims 1-25, wherein the carbon atom of R₂ that is bonded to X₂ is a secondary carbon.

30. The pharmaceutical composition according to any one of claims 1-25 and 29, wherein R₂ is heptadecan-9-yl.

31. The pharmaceutical composition according to any one of claims 1-30, wherein R₃ is alkyl_{(C≤24)} or substituted alkyl_{(C≤24)}.

32. The pharmaceutical composition according to any one of claims 1-31, wherein R₃ is alkyl_{(C≤24)}.

33. The pharmaceutical composition according to any one of claims 1-32, wherein R₃ is alkyl_{(C6-20)}.

34. The pharmaceutical composition according to any one of claims 1-33, wherein R₃ is n-alkyl_{(C6-20)}.

35. The pharmaceutical composition according to any one of claims 1-34, wherein R₃ is n-alkyl_{(C6-12)}.

36. The pharmaceutical composition according to any one of claims 1-35, wherein R₃ is n-hexane, n-heptane, n-octane, n-nonane, n-decane, n-undecane, or n-dodecane.

37. The pharmaceutical composition according to any one of claims 1-33, wherein the carbon atom of R₃ that is bonded to X₃ is a secondary carbon.

38. The pharmaceutical composition according to any one of claims 1-33 and 37, wherein R₃ is heptadecan-9-yl.

39. The pharmaceutical composition according to any one of claims 1-28, 31-33, 37, and 38, wherein R₂ is n-alkyl_{(C6-20)} and wherein the carbon of R₃ that is bonded to X₃ is a secondary carbon.

40. The pharmaceutical composition according to any one of claims 1-28 and 31-36, wherein R₂ and R₃ is n-alkyl_{(C6-20)}.

41. The pharmaceutical composition according to any one of claims 1-25, 29-33, 37, and 38, wherein the carbon of R₂ that is bonded to X₂ is a secondary carbon and wherein the carbon of R₃ that is bonded to X₃ is a secondary carbon.

42. The pharmaceutical composition according to claim 1, wherein the cationic ionizable lipid is further defined as: 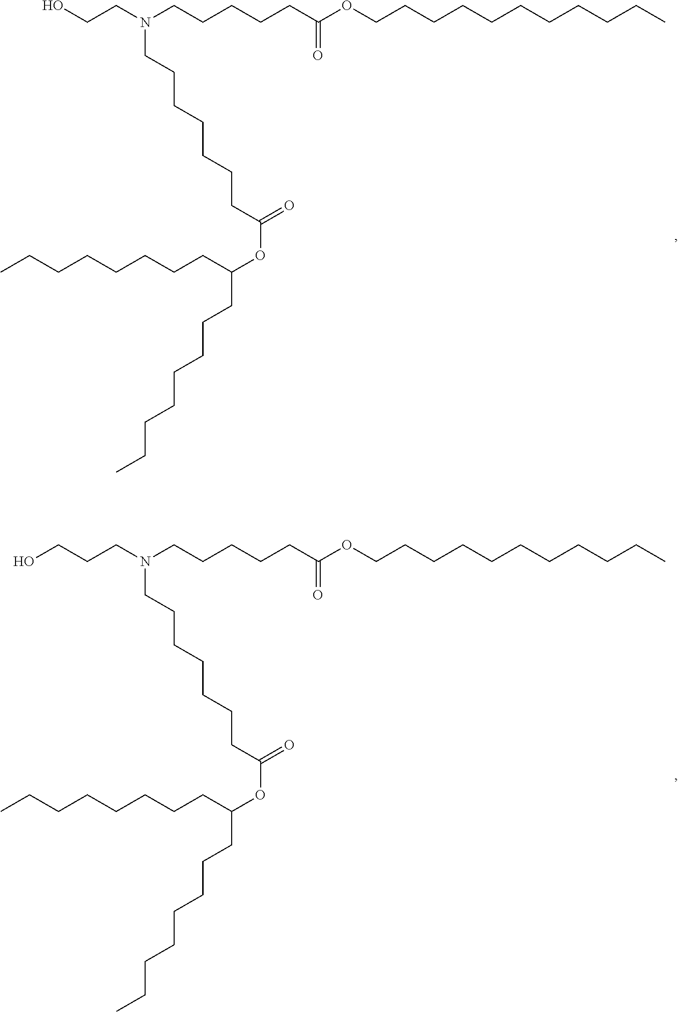 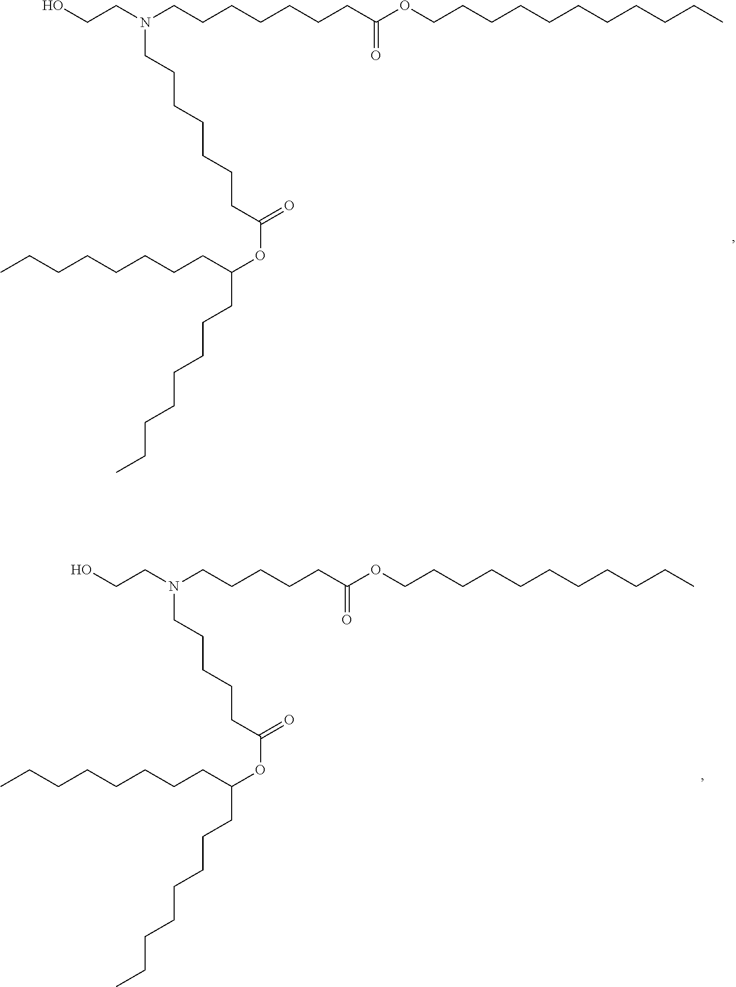 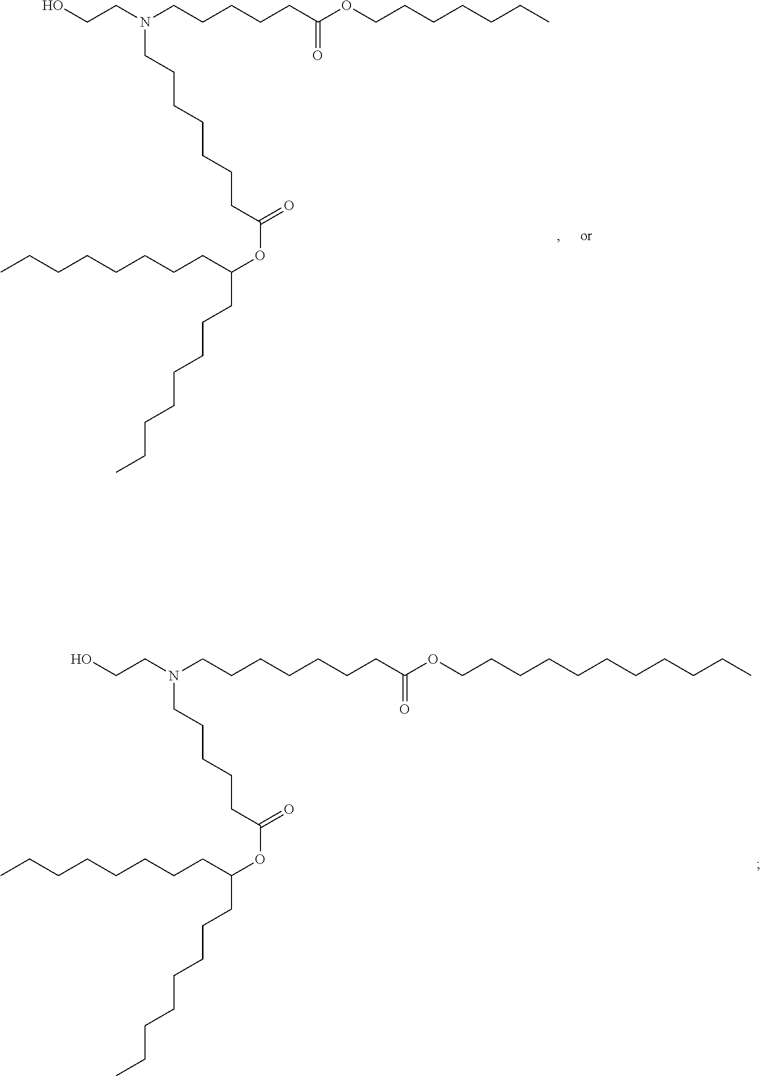
or a pharmaceutically acceptable salt thereof.

43. The pharmaceutical composition according to any one of claims 1-42, wherein the LNP comprises a molar ratio of the cationic ionizable lipid to the LNP of from about 0.3-0.7.

44. The pharmaceutical composition according to any one of claims 1-43, wherein the LNP comprises a molar ratio of the cationic ionizable lipid to the LNP of from about 0.4-0.6.

45. The pharmaceutical composition according to any one of claims 1-44, wherein the LNP comprises a molar ratio the cationic ionizable lipid to the LNP of about 0.45.

46. The pharmaceutical composition according to any one of claims 1-45, wherein the phospholipid comprises DOPE, DSPC, or DPPC.

47. The pharmaceutical composition according to any one of claims 1-46, wherein the phospholipid is DPPC.

48. The pharmaceutical composition according to any one of claims 1-47, wherein the LNP comprises a molar ratio of the phospholipid to the LNP of from about 0.02-0.4.

49. The pharmaceutical composition according to any one of claims 1-48, wherein the LNP comprises a molar ratio of the phospholipid to the LNP of from about 0.05-0.3.

50. The pharmaceutical composition according to any one of claims 1-49, wherein the LNP comprises a molar ratio of the phospholipid to the LNP of about 0.2.

51. The pharmaceutical composition according to any one of claims 1-50, wherein the PEG-lipid comprises DMG-PEG, DMPE-PEG, or DSPE-PEG.

52. The pharmaceutical composition according to any one of claims 1-51, wherein the PEG-lipid is DMPE-PEG.

53. The pharmaceutical composition according to any one of claims 1-52, wherein the LNP comprises a molar ratio of the PEG-lipid to the LNP of from about 0.005-0.03.

54. The pharmaceutical composition according to any one of claims 1-53, wherein the LNP comprises a molar ratio of the PEG-lipid to the LNP of from about 0.01-0.015.

55. The pharmaceutical composition according to any one of claims 1-54, wherein the LNP comprises a molar ratio of the PEG-lipid to the LNP of about 0.01.

56. The pharmaceutical composition according to any one of claims 1-55, wherein the LNP comprises DOPE and DMG-PEG, DOPE and DMPE-PEG, DOPE and DSPE-PEG, DSPC and DMG-PEG, DSPC and DMPE-PEG, DSPC and DSPE-PEG, DPPC and DMG-PEG, DPPC and DMPE-PEG, or DPPC and DSPE-PEG.

57. The pharmaceutical composition according to any one of claims 1-56, wherein the LNP comprises DPPC and DMPE-PEG.

58. The pharmaceutical composition according to any one of claims 1-55, wherein the sterol is cholesterol.

59. The pharmaceutical composition according to any one of claims 1-58, wherein the LNP comprises a molar ratio of the sterol to the LNP of from about 0.1-0.6.

60. The pharmaceutical composition according to any one of claims 1-59, wherein the LNP comprises a molar ratio of the sterol to the LNP of from about 0.15-0.5.

61. The pharmaceutical composition according to any one of claims 1-60, wherein the LNP comprises a molar ratio of the sterol to the LNP of about 0.34.

62. The pharmaceutical composition according to any one of claims 1-61, wherein the nucleic acid is a therapeutic nucleic acid.

63. The composition of claim 62, wherein the nucleic acid is siRNA, a miRNA, a pri-miRNA, a messenger RNA (mRNA), a cluster regularly interspaced short palindromic repeats (CRISPR) related nucleic acid, a single guide RNA (sgRNA), a CRISPR-RNA (crRNA), a trans-activating crRNA (tracrRNA), a plasmid DNA (pDNA), a transfer RNA (tRNA), an antisense oligonucleotide (ASO), a guide RNA, a double stranded DNA (dsDNA), a single stranded DNA (ssDNA), a single stranded RNA (ssRNA), and a double stranded RNA (dsRNA).

64. The pharmaceutical composition according to any one of claims 1-63, wherein the nucleic acid is an RNA.

65. The pharmaceutical composition according to any one of claims 1-64, wherein the nucleic acid comprises mRNA.

66. The pharmaceutical composition according to any one of claims 1-65, wherein the nucleic acid comprises siRNA.

67. The pharmaceutical composition according to any one of claims 1-66, wherein the nucleic acid is encapsulated in the LNP.

68. The pharmaceutical composition according to any one of claims 1-67, wherein the ratio of the CIL to the nucleic acid is from about 20:1 to about 1:5 by weight.

69. The pharmaceutical composition according to any one of claims 1-68, wherein the ratio of the CIL to the nucleic acid is from about 15:1 to about 1:3 by weight.

70. The pharmaceutical composition according to any one of claims 1-69, wherein the ratio of the CIL to the nucleic acid is about 11.3:1 by weight.

71. The pharmaceutical composition according to any one of claims 1-70, wherein the N/P ratio is from about 4-7.

72. The pharmaceutical composition according to any one of claims 1-71, wherein the N/P ratio is from about 5-6.

73. The pharmaceutical composition according to any one of claims 1-72, wherein the N/P ratio is about 5.7.

74. The pharmaceutical composition according to any one of claims 1-73, wherein the LNP has an average particle size of between about 50 nm and about 250 nm.

75. The pharmaceutical composition according to any one of claims 1-74, wherein the LNP has an average particle size of between about 50 nm and about 150 nm.

76. The pharmaceutical composition according to any one of claims 1-75, wherein the composition has an average particle size of about 100 nm.

77. The pharmaceutical composition according to any one of claims 1-75, wherein the composition has an average particle size of about 150 nm.

78. The pharmaceutical composition according to any one of claims 1-77, wherein the composition has a polydispersity index (PDI) of between about 0.01 and about 0.5.

79. The pharmaceutical composition according to any one of claims 1-78, wherein the composition has a polydispersity index (PDI) of between about 0.02 and about 0.4.

80. The pharmaceutical composition according to any one of claims 1-79, wherein the composition has a polydispersity index (PDI) of about 0.05.

81. The pharmaceutical composition according to any one of claims 1-79, wherein the composition has a polydispersity index (PDI) of about 0.3.

82. The pharmaceutical composition according to any one of claims 1-81, wherein the composition has a zeta potential of between about-0.5 mV and about-40 mV.

83. The pharmaceutical composition according to any one of claims 1-82, wherein the composition has a zeta potential of between about-0.5 mV and about-20 mV.

84. The pharmaceutical composition according to any one of claims 1-83, wherein the composition has a zeta potential of about-10 mV.

85. The pharmaceutical composition according to any one of claims 1-83, wherein the composition has a zeta potential of about-15 mV.

86. A nebulized composition in accordance with the pharmaceutical composition of any one of claims 1-85.

87. A method of treating a disease, disorder, injury, or infection comprising administering an effective amount of a pharmaceutical composition of any one of claims 1-86 to a subject.

88. The method of claim 87, wherein the disease is a genetic disease.

89. The method according to either one of claim 87 or claim 88, wherein the disease, injury, or infection is a lung disease, a lung injury, or a lung infection.

90. The method according to any one of claims 87-89, wherein the disease is a lung disease.

91. The method according to any one of claims 87-90, wherein the lung disease is interstitial lung disease, chronic obstructive pulmonary disease (COPD), asthma, cystic fibrosis (CF), pulmonary fibrosis, alpha-1 antitrypsin deficiency, or primary ciliary dyskinesia (PCD).

92. The method according to any one of claims 87-91, wherein the pharmaceutical composition is formulated for administration via inhalation.

93. The method according to any one of claims 87-92, wherein the subject is a mammal.

94. The method according to any one of claims 87-93, wherein the subject is a human.

95. A method of modulating the expression of a gene comprising delivering a nucleic acid to a cell, the method comprising contacting the cell with a pharmaceutical composition according to any one of claims 1-94 under conditions sufficient to cause uptake of the nucleic acid into the cell.

96. The method of claim 95, wherein the cell is contacted in vitro or ex vivo.

97. The method of claim 95, wherein the cell is contacted in vitro.

98. The method according to any one of claims 95-97, wherein the modulation of the gene expression is sufficient to treat a disease or disorder.

99. The method according to any one of claims 95-98, wherein the nucleic acid is an mRNA.
